(19) United States
(12) Patent Application Publication
(10) Pub. No.: US 2026/0043061 A1
(43) Pub. Date: Feb. 12, 2026

(54) ENZYMATICALLY MEDIATED REACTIVE CRYSTALLIZATION OF STEVIOL GLYCOSIDES

(71) Applicant: Arzeda Corp., Seattle, WA (US)

(72) Inventors: Michael Charles Milner COCKREM, Madison, WI (US); Kyle ROBERTS, San Marcos, CA (US)

(21) Appl. No.: 19/189,382

(22) Filed: Apr. 25, 2025

Related U.S. Application Data
(63) Continuation of application No. PCT/US2023/077888, filed on Oct. 26, 2023.
(60) Provisional application No. 63/419,612, filed on Oct. 26, 2022.
(60) Provisional application No. 63/429,342, filed on Dec. 1, 2022.
(60) Provisional application No. 63/443,104, filed on Feb. 3, 2023.

(51) Int. Cl. C12P 19/56; C07H 1/06; C07H 15/24; C12N 9/10

(52) U.S. Cl. CPC C12P 19/56; C07H 1/06; C07H 15/24; C12N 9/1062; C12Y 204/01013

(57) ABSTRACT

Methods to process steviol glycosides are provided herein. The method deploys enzymatically medicated reactive crystallization. Products comprising crystals produced by enzymatically mediated reactive crystallization are provided. A method to blend enzymatically mediated reactive crystallization products to provide high solubility is given. A method to generate highly soluble steviol glycoside products is provided whereby enzymatically medicated reactive crystallization mother liquors are evaporated and dried. The steviol glycoside crystals of the present disclosure increase specific sucrose equivalent value of the leaf, improve taste, lower cost, and improve quality.

### CROSS-REFERENCE TO RELATED APPLICATIONS

This application is a continuation of International Patent Application No. PCT/US2023/077888, filed Oct. 26, 2023, which claims priority to U.S. Provisional Patent Application No. 63/419,612, filed Oct. 26, 2022, U.S. Provisional Patent Application No. 63/429,342, filed Dec. 1, 2022, and U.S. Provisional Patent Application No. 63/443,104, filed Feb. 3, 2023, the contents of each of which are hereby incorporated in their entirety by this reference.

### REFERENCE TO AN ELECTRONIC SEQUENCE LISTING

The contents of the electronic sequence listing (ARZE_039_03WO_SeqList_ST26.xml; Size: 5,350 bytes; and Date of Creation: Oct. 24, 2023) are herein incorporated by reference in its entirety.

### TECHNICAL FIELD

The present disclosure relates to methods for enzymatically mediated reactive crystallization of steviol glycosides.

### BACKGROUND

Excess sugar consumption in the human diet is linked to reduced human health and increased healthcare costs. Replacing sugar with a low calorie, high-intensity sweetener may address these issues.

The plant species Stevia rebaudiana is grown for its sweet leaves, which have traditionally been used as a sweetener. Over ten different steviol glycosides are present in appreciable quantities in the leaf and most exhibit some degree of high intensity sweetness. In most varieties of Stevia rebaudiana, the principal steviol glycosides in the leaf are stevioside and rebaudioside A. To this end, Stevia leaves contain about 10% protein, 4% fats, 8% ash, 5-15% fiber, 60-70% carbohydrates. The carbohydrates include reducing sugars, fructooligosaccharides, polysaccharides, stevioside, rebaudiosides A & C, dulcoside, and other minor species. Additional leaf components include alkaloids, flavonoids, lutein, chlorophyll, polyphenols. Leaf extract, via e.g., hot water, can contain traces of all these leaf components.

The present disclosure describes compositions and methods for processing Stevia rebaudiana to generate soluble concentrations of steviosides.

### SUMMARY

The present disclosure provides methods to process steviol glycosides such as from Stevia leaf extract or fermentation, wherein the method further comprises an enzymatic reactive crystallization step. In embodiments, the present disclosure further provides compositions produced by such the methods described herein. In embodiments, the present disclosure further provides cocrystal compositions comprising two or more of rebaudioside M, rebaudioside D, and rebaudioside E.

In embodiments, the methods of the present disclosure can be used to generate and isolate steviol glycosides from Stevia leaf or from some fermentation broths via an aqueous solvent system, optionally without use of any solvents, resins, or membranes.

In embodiments, producing a composition comprising two or more of rebaudioside M, rebaudioside D, and rebaudioside E avoids the effects of bitterness commonly associated with steviol glycosides, specifically rebaudioside A and stevioside.

### BRIEF DESCRIPTION OF THE DRAWINGS

FIG. 1 is a flow diagram of a method for enzyme reactive crystallization (“ERX”) of a multicomponent mixture of steviol glycosides with enhanced solubility, resulting in crystalline steviol glycoside products.

FIG. 2 is a flow diagram, based on the ERX method of FIG. 1, with an additional cooling and/or evaporation step and/or additional recycle of mother liquor and/or seed crystals.

FIG. 3 is a flow diagram of an ERX method implemented with two sequential conversion steps for a feed. The feed comprises a multicomponent steviol glycoside mixture comprising at least rebaudioside D and stevioside to generate cocrystals rich in rebaudioside D as well cocrystals rich in rebaudioside M.

FIG. 4 is a flow diagram of the ERX method of FIG. 3, with an additional cooling and/or evaporation step.

FIG. 5 is a flow diagram of an ERX method implementing a recycle stream rich in rebaudioside A, wherein the recycle stream is blended in a mixer dissolver with a stream rich in stevioside to generate a multicomponent steviol glycoside mixture of enhanced solubility. A β-1,3-glycosylation reaction system can then be used to generate crystals rich in rebaudioside A.

FIG. 6 is a flow diagram of an ERX method implementing a recycle stream rich in rebaudioside A, wherein the recycle stream is blended in a mixer dissolver with a stream rich is stevioside so as to generate a multicomponent steviol glycoside mixture of enhanced solubility. A β-1,2-glycosylation and β-1,3-glycosylation reaction system can then be used to generate crystals rich in rebaudioside M.

FIG. 7 is an image of rebaudioside D-rich co-crystals produced by an ERX method described herein. The ERX method deploys RA50 at 15-liter reactor scale, vacuum oven drying, suspension in water, and light microscopy at 400× magnification.

FIG. 8 depicts crystallization kinetics of an ERX method as measured by volume percent suspended solids present in a 150-liter scale ERX run.

FIG. 9 is a graphical illustration of reaction kinetics of an ERX method as measured by conversion of stevioside and conversion of rebaudioside A in a 150-liter scale ERX run.

FIG. 10 is a graphical illustration of an overlay of the crystallization kinetics of FIG. 8 and the reaction kinetics from FIG. 9, illustrating a time-lag or time-offset between reaction and crystal formation. The square and triangle tracers in FIG. 10 show conversion of stevioside and Reb A to a product measured by HPLC. The open circles show the amount or volume of suspended solids (VSS).

FIG. 11 is a graphical illustration of a theoretical calculation of sucrose equivalent, per kilogram of harvested leaf, showing substantial value in effective sweetness obtainable by enzymatically upgrading the leaf extract. Steviol glycosides containing rhamnosyl moieties, which are excluded from this graphic for visual clarity, have significantly lower sucrose equivalence.

FIG. 12 is a UV-Vis spectrophotometric scan of crystals produced at laboratory scale by an ERX method, showing excellent purity in regard to absence of impurities that adsorb at 254 nm, 270 nm, 280 nm, and 315 nm.

FIG. 13 shows the enzymatic reactions carried out by a beta-1,2-glycosyltransferase and a beta-1,3-glycosyltransferase to convert stevioside to rebaudioside M.

FIG. 14 shows the stevioside molecule and indicates to which carbon (C13, (G) reaction, or C19 (E) reaction) a beta-1,2-glycosyltransferase and a beta-1,3-glycosyltransferase can attach additional glucose units during the conversion to rebaudioside M.

FIG. 15 is a flow diagram of an ERX method implementing a purification of the ERX product via heating the reaction slurry to dissolve the ERX product and provide formation of a hot break and removal of the hot break followed by crystal reforming via cooling crystallization.

FIG. 16 is a flow diagram of an ERX method implementing a purification of the ERX product via heating the reaction slurry to dissolve the ERX product and provide formation of a hot break and removal of the hot break followed by crystal reforming via evaporative crystallization.

FIG. 17 is a flow diagram of an ERX method implementing a purification of the ERX product via dissolving the recovered ERX crystals in a solvent, with or without heating followed, by crystal reforming via either cooling or evaporative or combined mode crystallization. No hot break or solvent break is removed.

FIG. 18 is a flow diagram of an ERX method implementing a purification of the ERX product via dissolving the recovered ERX crystals in a solvent, with or without heating, to provide formation of a hot break or solvent break and removal of the hot or solvent break followed by crystal reforming via either cooling or evaporative or combined mode crystallization.

FIG. 19 is a flow diagram of an ERX method implementing a purification of the ERX product via sequential combination of methods shown in FIG. 15 and in FIG. 18.

FIG. 20 shows a single component feed to ERX with solubility of the feed enhanced by temperature.

FIG. 21 is a graphical representation of mother liquor recycling. FIG. 21 illustrates high-performance liquid chromatography results showing a RXN1 control having 86% Reb M at hour 3, which can be compared with a RXN2 sample, with recycled mother liquor, having a slower rate of Reb M formation. Regarding a RXN2 sample, with recycled mother liquor, Reb M reached 31% at hour 6, and Reb AM, Reb D, and Reb E reached 47% at hour 6. Regarding the RXN1 control, Reb AM, Reb D, and Reb E reached 1% by hour 3.

FIG. 22 is a tabular representation of steviol glycoside generation at a variety of timepoints in the presence and/or absence of glycosyltransferases, sucrose synthases, ADP, and/or sucrose.

FIG. 23 is a tabular representation of steviol glycoside generation at a variety of timepoints in the presence and/or absence of recycled glycosyltransferases, sucrose synthases, ADP, and/or sucrose.

### DETAILED DESCRIPTION

Non-limiting examples of various aspects and variations of the invention are described herein and illustrated in the accompanying drawings.

### Abbreviations

Abbreviations used herein are given in Table 1.

TABLE 1
Abbreviations

| | Abbreviation |
|---|---|
| | Herein |
| Enzyme Reactive Crystallization | ERX |
| Sucrose synthase | SuSy |
| β-1,3-glycosyltransferase | B13GT |
| β-1,2-glycosyltransferase | B12GT |
| Nucleotide diphosphate | NDP |
| Adenosine diphosphate | ADP |
| Stevia leaf extract with >95% steviol glycosides, >50% | RA50 |
| rebaudioside A and >30% stevioside by mass | |
| Stevia leaf extract with >95% steviol glycosides, >20% | RA20 |
| rebaudioside A and >60% stevioside by mass | |
| Stevia leaf extract with >95% steviol glycosides, >40% | RA40 |
| rebaudioside A and >40% stevioside by mass | |
| Stevia leaf extract with >95% steviol glycosides, >60% | RA60 |
| rebaudioside A and >20% stevioside by mass | |

### Definitions

The term “a” or “an” refers to one or more of that entity, i.e. can refer to plural referents. As such, the terms “a,” “an.” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs (BLOSUM62 matrix; Gap Open Penalty −1; Gap Extend Penalty −1; Gap Align set to True). Unless noted otherwise, the term “sequence identity” in the claims refers to sequence identity as calculated by Clustal Omega® using default parameters. Clustal Omega uses the HHalign algorithm and its default settings as its core alignment engine. The algorithm is described in Soding, J. (2005) ‘Protein homology detection by HMM-HMM comparison’. Bioinformatics 21, 951-960. The default transition matrix is Gonnet, gap opening penalty is 6 bits, gap extension is 1 bit.

As used herein, “biocatalysis” or “biocatalytic” refers to the use of natural catalysts, such as protein enzymes, to perform chemical transformations on organic compounds. Biocatalysis is alternatively known as biotransformation or biosynthesis. Both isolated and whole-cell biocatalysis methods are known in the art. Biocatalyst protein enzymes can be naturally occurring or recombinant proteins.

As used herein, the terms “polynucleotide” or “nucleic acid” are used interchangeably, unless indicated by context, and is used to refer to a polymeric form of nucleotides of any length, either ribonucleotides or deoxyribonucleotides, typically DNA.

As used herein, “expression” refers to either or both steps, depending on context, of the two-step process by which polynucleotides are transcribed into mRNA and the transcribed mRNA is subsequently translated into a polypeptides.

The regulatory elements, e.g. enhancers and promoters, may be “homologous” or “heterologous.” A “homologous” regulatory element is one which is naturally linked with a given polynucleotide in the genome; for example, it may be the promoter found natively in the organism upstream of the encoded polypeptide. A “heterologous” regulatory element is one which is placed in juxtaposition to a polynucleotide by means of recombinant molecular biological techniques but is not a combination found in nature. Often, promoters, enhancers and other regulatory elements are heterologous so as to facilitate expression of a polypeptide in a host cell other than one in which a polypeptide naturally occurs. Thus, “heterologous expression”, as used herein, refers to producing an mRNA and/or a polypeptide in a host cell, such as a microorganism, where the polynucleotide is not found naturally or one or more regulatory elements are not naturally found operably linked to the polynucleotide in the host cell.

As used herein, the terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “microbes” of lists and figures present in the disclosure. This characterization can refer to not only the identified taxonomic genera but also the identified taxonomic species, as well as the various novel and newly identified or designed strains of any organism in said tables or figures. The same characterization holds true for the recitation of these terms in other parts of the Specification, such as in the Examples.

As used herein, “enzyme reactive” refers to the use of natural catalysts, such as protein enzymes, to perform chemical transformations on organic compounds. Biocatalysis is alternatively known as biotransformation or biosynthesis. Biocatalyst protein enzymes can be naturally occurring or recombinant proteins.

The term “polypeptide” is used here to refer to a molecule of two or more subunits of amino acids linked by peptide bonds. Typically, though not always, the polypeptides contain several hundred amino acids; for example, about 400 to about 900 amino acids.

As used herein, the term “steviol glycoside(s)” refers to a glycoside of steviol, including, but not limited to, naturally occurring steviol glycosides, e.g. steviol-13-O-glucoside, steviol-19-O-glucoside, rubusoside, steviol-1,2-bioside, steviol-1,3-bioside, rubusoside, dulcoside B, dulcoside A, rebaudioside B (“Reb B”), rebaudioside G (“Reb G”), stevioside, rebaudioside C (“Reb C”), rebaudioside F (“Reb F”), rebaudioside A (“Reb A”), rebaudioside I (“Reb I”), rebaudioside E (“Reb E”), rebaudioside E2 (“Reb E2”), rebaudioside AM (“Reb AM”), rebaudioside H (“Reb H”), rebaudioside L (“Reb L”), rebaudioside K (“Reb K”), rebaudioside J (“Reb J”), rebaudioside M (“Reb M”), rebaudioside D (“Reb D”), rebaudioside N (“Reb N”), rebaudioside O (“Reb O”), rebaudioside Q (“Reb Q”), synthetic steviol glycosides, e.g. enzymatically glycosylated steviol glycosides and combinations thereof.

As used herein, rebaudioside X may refer to a high-purity variation of rebaudioside M.

As used herein, “multicomponent mixture of steviol glycosides” refers to a mixture wherein there are at least two different steviol glycosides.

As used herein, “enhanced solubility” refers to a solution, typically aqueous, wherein the weight concentration of components of interest, for instance a multicomponent mixture of steviol glycosides, is greater than the solubility for individual, pure component steviol glycosides.

As used here, the “specific sucrose equivalent value” of the leaf is the mass (kilogram, “kg”) of sucrose equivalent than can be derived from a kg of leaf (dry basis) as harvested.

As used herein, “hot break” refers to impurities that become insoluble upon heating. The hot break may also incorporate insoluble impurities that are trapped in the hot break solids.

As used herein, “solvent break” refers to impurities that become insoluble upon dissolving in a solvent other than water, such as alcohol (e.g. ethanol, methanol, isopropanol). A solvent break step may be followed by heating, and the break or accumulation of impurities is then increased.

As used herein, “re-crystallization” refers to a second crystallization step that is subsequent to a first crystallization step. As used herein, “cooling crystallization” refers to crystallization wherein the temperature is lowered. As used herein, “evaporative crystallization” refers to crystallization by removal of solvent, thereby increasing concentration. The solvent may be water or another solvent such as ethanol.

As used herein, “starting composition” refers to any composition (generally an aqueous solution) containing one or more steviol glycosides, where the one or more steviol glycosides serve as the substrate for the biotransformation.

As used herein, “NDP” refers to a nucleotide diphosphate. The nucleotide diphosphate can be any form of adenosine diphosphate (ADP), uridine diphosphate (UDP), cytidine diphosphate (CDP), thymidine diphosphate (TDP), and guanosine diphosphate (GDP). “ADP” refers to any form of adenosine diphosphate, including but not limited to anhydrous adenosine 5′-diphosphate, adenosine 5′-diphosphate disodium salt, adenosine 5′-diphosphate monopotassium salt dihydrate, adenosine 5′-diphosphate sodium salt, adenosine-5′-diphosphate disodium salt dihydrate, adenosine 5′-diphosphate bis(cyclohexylammonium) salt.

Note that “glycosyl” refers to all sugar residues. Glucosyl is specific to glucose residues.

A co-crystal is defined as a homogenous crystalline material made of two or more molecules in definite stoichiometric amounts held together by non-covalent forces. The FDA further defines that the pKa difference between the co-formers should be less than 1, thus indicating a non-ionic species and minimal proton sharing. A eutectic is defined as the point of temperature and molar ratio where two solids become perfectly miscible and is recognizable as the minimum coherent point on the phase diagram. Both co-crystals and eutectics exhibit similar short-range ordering. However, co-crystals differ in that they exhibit long range order also. Whether a co-crystal or a eutectic will form depends on relative strengths of enthalpic advantage of packing relative to entropic loss due to ordering.

Generally, the present disclosure provides methods for reacting a mixture of one or more steviol glycosides with one or more glycosyltransferases, thereby generating new mixtures from which steviol glycosides crystallize or precipitate. To this end, the enzymatically mediated reactive crystallization (ERX) methods described herein involve a feed composition (or starting composition) comprising one or more steviol glycosides that are soluble at modest to high concentrations. The starting composition is contacted by enzymes within a reaction composition that convert at least part of the starting composition to create a new species (or target steviol glycoside(s)) that has lower solubility. As a result, at least portion of the converted, target steviol glycoside(s) will crystallize or precipitate. In this way, the target steviol glycoside(s) can be recovered by, for instance, simple filtration instead of other, intensive methods requiring e.g., a resin adsorption/desorption or crystallization, which is more complex and with lower throughput. The steviol glycoside component(s) of the starting composition serves as a substrate(s) to produce the target steviol glycoside(s), as will described herein. The target steviol glycoside(s) differs chemically from its corresponding substrate steviol glycoside(s) by the addition of one or more sugar monomer units. In some embodiments, the target steviol glycoside(s) differs chemically from its corresponding substrate steviol glycoside(s) by the addition of one or more glucose monomer units.

In an embodiment, the enzyme reaction involves reacting the starting steviol glycoside composition with an NDP-sugar, and one or more NDP-glycosyltransferase polypeptides, thereby producing a mixture of precipitated steviol glycosides and, optionally, soluble steviol glycosides. The resulting precipitated and optional soluble steviol glycosides can comprise target steviol glycosides and, optionally, unreacted steviol glycosides. In an embodiment, the enzyme reaction involves reacting the starting steviol glycoside composition with an NDP-glucose and one or more NDP-glycosyltransferase polypeptides, thereby producing a mixture of precipitated steviol glycosides and, optionally, soluble steviol glycosides. The resulting precipitated and soluble steviol glycosides can be composed of target steviol glycosides and, optionally, unreacted steviol glycosides.

Embodiments of the present disclosure utilize a SuSy NDP-glucose recycling system combined with one or more NDP-glycosyltransferase polypeptides to convert the starting steviol glycoside composition to the target steviol glycoside composition. The enzyme reaction may involve reacting the starting composition of steviol glycosides with a sugar monomer source, NDP, one or more NDP-glycosyltransferase polypeptides, and an enzymatic NDP-sugar recycling system, thereby producing a slurry of precipitated steviol glycosides and, optionally, soluble steviol glycosides. The resulting precipitated and optional soluble steviol glycosides can be composed of target steviol glycosides and, optionally, unreacted steviol glycosides. The enzyme reaction may involve reacting substrate steviol glycosides, sucrose, and NDP with one or more NDP-glycosyltransferase polypeptides and a sucrose synthase, thereby producing a slurry of precipitated steviol glycosides and, optionally, soluble steviol glycosides. The resulting precipitated and optionally soluble steviol glycosides can be composed of target steviol glycosides and, optionally, unreacted steviol glycosides.

A starting composition of Reb A and/or stevioside may be reacted with sucrose, NDP, a B12GT polypeptide, and a SuSy polypeptide to produce Reb D and Reb E, respectively. A starting composition of RA50 may be reacted with sucrose. ADP, an engineered B12GT polypeptide and an engineered SuSy polypeptide to produce a precipitated steviol glycoside composed primarily of RebD and soluble mother liquor composed primarily of Reb E.

A starting composition of Reb A and/or stevioside may be reacted with sucrose, NDP, a B12GT polypeptide, a B13GT polypeptide and a SuSy polypeptide to produce Reb M. In another embodiment, a starting composition of RA50 is reacted with sucrose, ADP, an engineered B12GT polypeptide, an engineered B13GT, and an engineered SuSy polypeptide to produce a precipitated steviol glycoside composed primarily of Reb M and soluble mother liquor composed primarily of Reb M.

As introduction to rebaudiosides, the major steviol glycosides present in a Stevia rebaudiana leaf have a taste profile that can be undesirable due to a lingering bitter aftertaste. There is a need to improve the taste profile to increase applicable uses of the sweetener. Furthermore, higher molecular weight steviol glycosides, such as rebaudioside D and rebaudioside M, have greater sweetness per unit mole with reduced lingering bitterness. Enzymatic glycosylation of harvested steviol glycosides can increase the value of Stevia crop simply by increasing the sweetness equivalent per unit harvested.

Many steviol glycosides have only glucosyl residues derived from glucose, whereas some will have one or more other sugar residues such as xylose and rhamnose, among others. For example, rebaudioside F has three glucosyl residues and one xylosyl residue while rebaudioside C has three glucosyl residues and one rhamnosyl residue.

Enzymatic reactions give linear or branched chains of glucosyl or other sugar residues with as many as 9 residues. Steviol glycosides with glucosyl residues are shown in Table 2, wherein the un-glucosylated core molecule is steviol. In Stevia leaf, levels of steviol may be very low and often are not detected by usual analytical methods. Moreover, species of Stevia leaf with five or more glucosyl residues may not be present or present only at very low concentrations and thus may not be detectable with usual measurements.

TABLE 2
Glucosyl Residues

| | Glucosyl | | | | |
|---|---|---|---|---|---|
| Formula | Residues | MW | Examples | | |
| C₂₀H₃₀O₃ | 0 | 318.5 | Steviol | | |
| C₂₆H₄₀O₈ | 1 | 480.6 | Steviol- | | |
| | | | monoside | | |
| C₃₂H₅₀O₁₃ | 2 | 642.7 | Steviobioside | | |
| C₃₈H₆₀O₁₈ | 3 | 804.9 | Stevioside | Reb B | |
| C₄₄H₇₀O₂₃ | 4 | 967.0 | Reb A | Reb E | Reb E2 |
| C₅₀H₈₀O₂₈ | 5 | 1,129.2 | Reb D | Reb I | Reb AM |
| C₅₆H₉₀O₃₃ | 6 | 1,291.3 | Reb M | Reb M2 | |
| C₆₂H₁₀₀O₃₈ | 7 | 1,453.4 | | | |
| C₆₈H₁₁₀O₄₃ | 8 | 1,615.6 | | | |
| C₇₄H₁₂₀O₄₈ | 9 | 1,777.7 | | | |

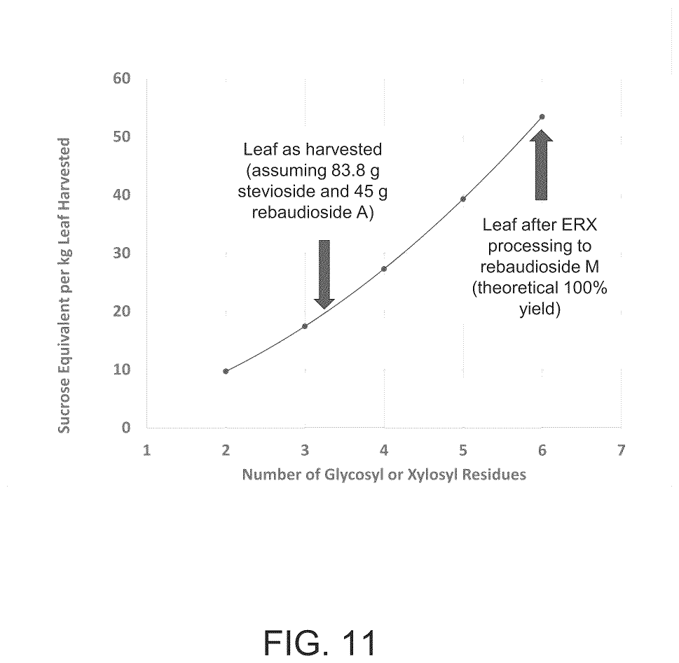

The value of the Stevia leaf extract can be increased by improving taste, reducing costs, and improving purity of steviol glycosides. Consider, for example, that 1 kilogram (kg) of Stevia leaf (dry basis) contains 83.85 grams (g) of stevioside, 45.15 g of rebaudioside A, and a smaller amount of additional steviol glycosides. The total stevioside and rebaudioside A in said 1 kg of leaf comprises a total of 0.15 mole, which has a sucrose equivalence (SE) of approximately 20.6 kg SE per kg of leaf. Processing the same Stevia leaf using the methods described in the present disclosure upgrades the 0.15 mole to give 0.15 mole of rebaudioside M, which is the theoretical case at 100% molar yield. This now has a SE of 53.4 kg SE per kg of leaf. This is illustrated in FIG. 11, which shows the dependence of measured sucrose equivalent on the number of glycosyl or xylosyl moieties present in the steviol glycoside. The process here has increased the effective yield of SE per kg of leaf harvested by 2.6-fold (53.4/20.6). This provides a substantial economic benefit.

Pure steviol glycosides are sparingly soluble in water. Solubility measurements may reflect the true thermodynamic solubility or a short-term kinetic solubility. The kinetic solubility value occurs when an isomeric form has been prepared that is not thermodynamically stable at long time scales yet has higher solubility at short times scales. A few steviol glycoside species have a free carboxylic acid group. These species tend to have higher solubility. Nevertheless, steviol glycosides are generally poorly soluble in water, as shown in Table 3 (certain measurements are for species that may not be completely pure).

TABLE 3
Solubility of Select Steviol Glycosides at Room Temperature

| Steviol Glycoside | wt % |
|---|---|
| Reb A anhydrous | 0.80% |
| Reb A 97% | 0.80% |
| Reb A hydrate | 0.10% |
| Reb A | 0.46% |
| Stevioside | 0.44% |
| Reb C | 0.20% |
| Reb D | 0.10% |
| Reb M | 0.05% |

Typically, steviol glycosides can be recovered from Stevia rebaudiana leaf by hot water extraction, various clarification steps, use of a resin adsorption step, and/or an alcohol desorption step. This may be followed by additional aqueous and non-aqueous recrystallization steps, as well as additional resin adsorption and desorption steps. Because of the poor solubility of pure steviol glycosides in water, these efforts to generate rebaudiosides from leaf extracts are limited to low reaction concentrations and difficulties in isolating converted rebaudiosides from the “mother liquor”. Therefore, there is a need to provide improved processes that increase yields of converted rebaudiosides and improve isolation and purification of the converted rebaudiosides. Such improved processes can minimize or avoid usage of resins and solvents, as well as minimize volumes of water to be processed. Further, such improved processes address the need for more cost-effective processing methods for enzymatic glycosylation by allowing for higher operating concentrations (and thus smaller reactor volumes).

To this end, the present disclosure provides methods for efficiently processing steviol glycosides by leveraging reactive crystallization to improve rebaudioside isolation and purification. The methods described herein leverage the enhanced solubility of stevioside together with rebaudioside A to enhance reaction concentrations, which naturally improves the taste profile of the target composition.

The present disclosure deploys a reactive crystallization step to aid in processing steviol glycosides. When supersaturation of a crystallizing compound is created by chemical reaction, the operation is known as reactive crystallization. Reactive crystallization, or precipitation, involves a reaction between starting materials to form a solute which crystallizes into a solid product. For instance, in the example of steviol glycosides herein, a reaction between a glycosyltransferase, or other enzyme capable of adding a sugar monomer to a steviol glycoside, and a steviol glycoside can produce a new steviol glycoside that has lower solubility in the solution compared to the precursor steviol glycoside and, therefore, precipitates out as a crystal. This may be the case, for example, when a B13GT adds a sugar monomer to a Reb D, which converts it to a Reb M, which precipitates out of solution. Examples of industrial relevance include the liquid-phase oxidation of para-xylene to terephthalic acid.

In some variations, the present disclosure describes methods for generating co-crystals as the enzymatically mediated reactive crystallization crystal product. A co-crystal, as introduced above, is defined as a homogenous crystalline material made of two or more molecules in definite stoichiometric amounts held together by non-covalent forces. The FDA further defines that the pKa difference between the co-formers should be less than 1, thus indicating a non-ionic species and minimal proton sharing. If one of the starting materials is normally a liquid, then the combined substance is called a solvate. As noted herein, a eutectic is defined as the point of temperature and molar ratio where two solids become perfectly miscible and is recognizable as the minimum coherent point on the phase diagram. Both co-crystals and eutectics exhibit similar short-range ordering. However, co-crystals differ in that they exhibit long range order also. Whether a co-crystal or a eutectic will form depends on relative strengths of enthalpic advantage of packing relative to entropic loss due to ordering. Sufficient thermodynamic models may permit predictive calculation via minimization of the free energy of the crystal phase mixture. The co-crystalline phase can be identified and characterized by thermal and diffraction analyses including single-crystal X-ray diffraction. The energies of intermolecular interactions in the crystal can be calculated by solid-state DFT and PIXEL methods. Such calculation schemes show total energy of intermolecular interactions. Thermodynamic functions of co-crystal formation can be estimated from the solubility of the co-crystal and the corresponding solubility of the pure compounds at various temperatures. The co-crystal formation process driving force can be estimated as Gibbs energy [kJ·mol⁻¹], and endothermic or exothermic enthalpy of formation, as well as entropy, can be estimated. Sufficient thermodynamic models, however, are not available for systems containing steviol glycoside components.

In an ideal coherent, theoretical-based approach for the design of reactive crystallization systems, enzyme reaction kinetics, diffusion effects, and crystallization kinetics, and mass transfer are considered. A good description will include reaction, mass transfer, dissolution Damköhler numbers, and the nucleation and growth numbers. From this, the impact of the individual steps and their effect on crystal-size distribution and the crystallizer productivity can be estimated. To this end, kinetic models for enzyme thermal and non-thermal inactivation must also be considered. Secondary crystallizer processes such as aging, agglomeration, and breakage can also occur, complicating the design of a reactive crystallizer.

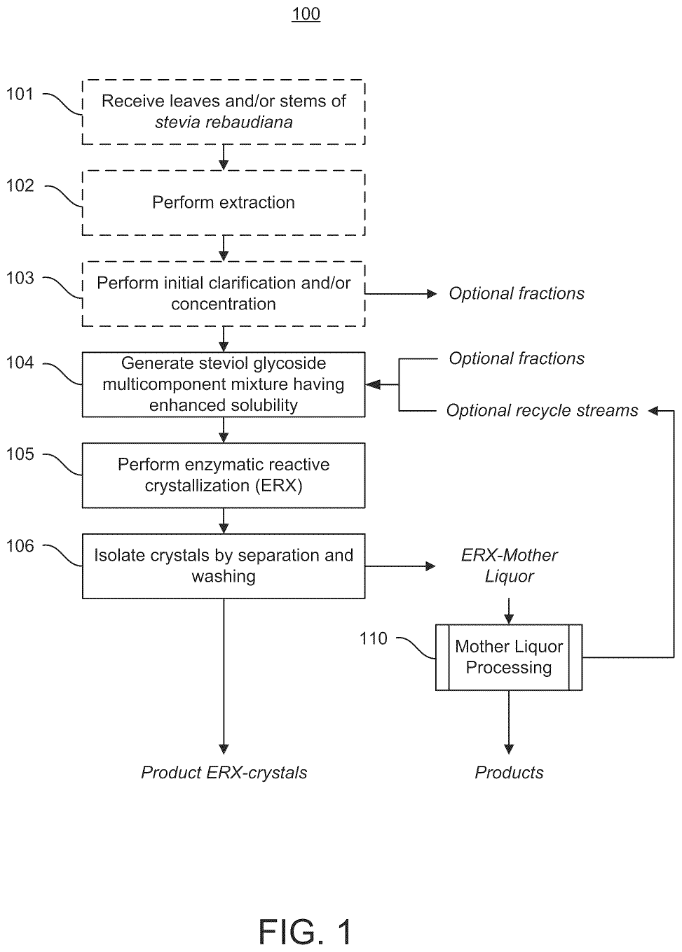

Referring now to the Drawings, a method of generating and isolating steviol glycoside crystals will be described with reference to method 100 of FIG. 1. Dashed boxes indicate optional steps.

At optional step 101 of method 100, leaves and/or stems of the Stevia rebaudiana may be received. At optional steps 102 and 103 of method 100, the leaves and/or stems of the Stevia rebaudiana may be processed to extract steviol glycosides and to perform initial clarification and/or concentration thereof. Extraction may be performed by batch parabolic trough with a helical mixer, a batch rotating cylindrical extractor, a batch percolation extractor, a horizontal continuous moving belt extractor, a horizontal countercurrent tubular screw extractor, a vertical countercurrent tubular screw extractor, a segmented rotating basket extractor, and a pressurized condensing stream extractor. Clarification can include thermal coagulation of proteins and impurities, diatomaceous earth treatment, ion exchange, and chemical coagulation using calcium carbonate and/or ferric chloride. Concentration can include membrane processing (e.g., nanofiltration), evaporation, and adsorption of one or more steviol glycosides onto a solid adsorbent followed by elution with an alcohol and removal to give a concentrated leaf extract. Fractions may be obtained during this processing and may be reintroduced at step 105, as shown in FIG. 1.

At step 104 of method 100, a starting composition, or steviol glycoside mixture having enhanced solubility, can be generated from the processed Stevia leaf extract. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

In embodiments, the starting composition may comprise a purified substrate steviol glycoside. For example, the starting composition may comprise greater than 50%, greater than 60%, greater than 70%, greater than 80%, greater than 85%, greater than 90%, greater than 91%, greater than 92%, greater than 93%, greater than 94%, greater than 95%, greater than 96%, greater than 97%, greater than 98%, greater than 99%, or greater than 99.6% of one or more steviol glycosides by weight on an anhydrous basis. In another embodiment, the starting composition comprises a partially purified substrate steviol glycoside composition. For example, the starting composition contains greater than 0.5%, greater than 1%, greater than 2%, greater than 3%, greater than 4%, greater than 5%, greater than 10%, greater than 20%, greater than 30%, greater than 40%, or greater than 50%, of one or more substrate steviol glycosides by weight on an anhydrous basis. In another embodiment, the substrate steviol glycoside is purified rebaudioside A, or isomers thereof. In a particular embodiment, the substrate steviol glycoside contains greater than 99% rebaudioside A. or isomers thereof, by weight on an anhydrous basis. In another embodiment, the substrate steviol glycoside comprises partially purified rebaudioside A. In a particular embodiment, the substrate steviol glycoside contains greater than 1%, 5%, 10%, 20%, 30%, 40%, 50%, 60%, 70%, 80% or 90% rebaudioside A by weight on an anhydrous basis. In yet another embodiment, the substrate steviol glycoside comprises purified stevioside, or isomers thereof. In a particular embodiment, the substrate steviol glycoside contains greater than 99% stevioside, or isomers thereof, by weight on an anhydrous basis. In another embodiment, the substrate steviol glycoside comprises partially purified stevioside. In a particular embodiment, the substrate steviol glycoside contains greater than 10%, 20%, 30%, 40%, 50%, 60%, 70%, 80% or 90% stevioside by weight on an anhydrous basis.

At step 105 of method 100, the starting composition can be reacted with a reaction composition to perform enzymatic reactive crystallization (ERX). The ERX may be performed in batches or semicontinuous.

In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme is added to the reaction composition at the start of the reaction. For instance, when the at least one enzyme comprises a β12GT and a β13GT, the glycosyltransferase may be provided together at the start of the reaction. In another embodiment, a first portion of the at least one enzyme can be added at the start of the reaction and one or more additional portions of the at least one enzyme can be added during the time course of the reaction. In embodiments, the aqueous reaction mixture comprises one or more of sodium chloride, sodium acetate, potassium chloride, sodium sulfate, and sodium phosphate to aid in maintaining enzyme solubility.

In embodiments, the at least one enzyme does not produce appreciable products greater than molecular weight of about 1300 g/mol. For instance, the enzyme products may have a molecular weight between less than about 1300 g/mol, less than about 1200 g/mol, less than about 1100 g/mol, less than about 1000 g/mol, less than about 900 g/mol, less than about 800 g/mol, less than about 700 g/mol, less than about 600 g/mol, less than about 500 g/mol, less than 400 g/mol, less than about 200 g/mol, and less than about 100 g/mol.

In embodiments, the at least one enzyme can be provided in any suitable form, including free, immobilized, or as a whole cell system. The degree of purity of the at least one enzyme may vary. For example, it may be provided as a crude, semi-purified, or purified enzyme preparation(s). In one embodiment, the at least one enzyme is free. In another embodiment, the at least one enzyme is immobilized to a solid support, for example on an inorganic or organic support. In some embodiments, the solid support is derivatized cellulose, glass, ceramic, methacrylate, styrene, acrylic, a metal oxide, or a membrane. In some embodiments, the at least one enzyme is immobilized to the solid support by covalent attachment, adsorption, cross-linking, entrapment, or encapsulation. In some embodiments, the at least one enzyme is provided in the form of a whole cell system, for example as a living fermentative microbial cell, or as dead and stabilized microbial cell, or in the form of a cell lysate.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use an NDP-sugar, such as ADP-glucose, as a sugar donor to modify steviol glycosides by adding sugars thereto. In embodiments, the NDP-sugar may comprise one of galactose, glucose, xylose, glucosamine, galactosamine, glucuronic acid, galactofuranose, mannose, fucose, rhamnose, acetylneuraminic acid, and mannooctanoic acid.

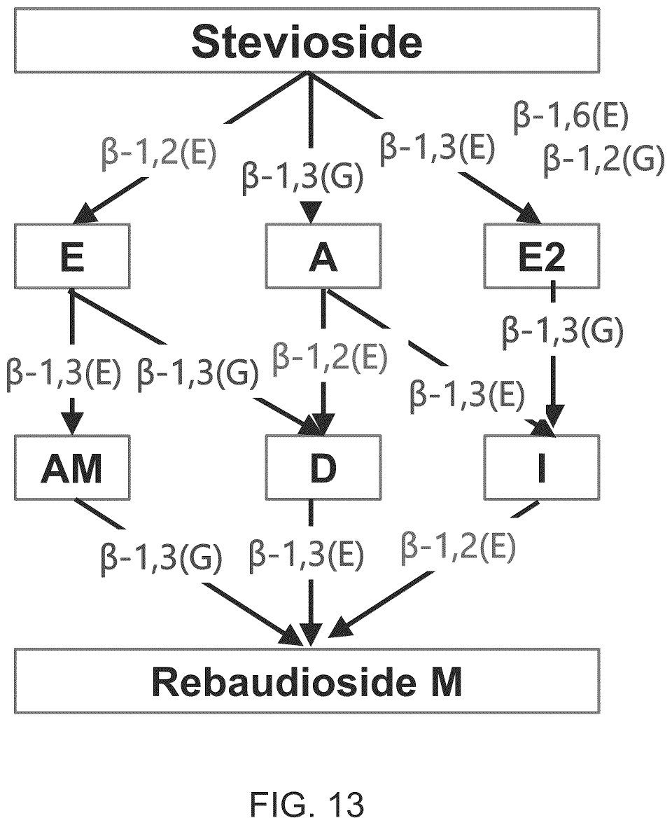

FIG. 13 is a flow diagram illustrating the role of each of B12GT and B13GT within a stevioside to Reb M pathway. In embodiments, the B12GT may be add a beta-linked glucose monomer to the C2 of the 13-O-glucose and/or 19-O-glucose of a steviol glycoside substrate. For example, the B13GT may use an ADP-glucose sugar donor convert Stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may add a beta-linked glucose monomer to the CY of the 13-O-glucose and/or 19-O-glucose of a steviol glycoside substrate. For example, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

In embodiments, when the reaction composition comprises at least one glycosyltransferase enzyme, reacting the starting composition and the reaction composition results in the addition of

In embodiments, the at least one enzyme comprises one or more sucrose synthases. The one or more sucrose synthases may be a wild type sucrose synthase, a non-natural, engineered sucrose synthase, a polypeptide having sucrose synthase activity, or a combination thereof. In embodiments, the one or more sucrose synthases (SuSy) may use a sucrose sugar donor to convert an NDP and sucrose to form NDP-glucose and fructose. For example, the one or more SuSy may convert ADP and sucrose to ADP-glucose and fructose. In embodiments, the SuSy may be a non-natural, engineered SuSy as described in International Patent Application No. PCT/US2022/016820, which is incorporated herein by reference in its entirety.

In embodiments, the at least one enzyme comprises a wild type, a non-natural, or engineered phosphoglucomutase or polypeptide having phosphoglucomutase activity. In embodiments, the reaction composition may comprise pyrophosphate reagent and/or disodium pyrophosphate.

In embodiments, the reaction composition used in the reaction at step 105 of method 100 may be an aqueous medium. The reaction composition may comprise at least one buffer selected from the group including acetate buffer, citrate buffer, HEPES, and phosphate buffer. In embodiments, the buffer may have a pH of between about 4 and about 10, about 4.5 and about 9, about 5 and about 8, about 5.5 and about 7, and about 6 and about 6.5.

In embodiments, the reaction at step 105 of method 100 can be performed at a temperature between about 10° C., and about 80° C., between about 10° C., and about 20° C., between about 20° C., and about 30° C., between about 30° C., and about 40° C., between about 40° C. and about 50° C., between about 50° C., and about 60° C., between about 60° C., and about 70° C., and between about 70° C., and about 80° C. In an embodiment, the reaction can be performed at about 60° C.

In embodiments, the reaction at step 105 of method 100 can be performed for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the reaction can be performed for a duration of about 6 hours.

In embodiments, the reaction at step 105 of method 100 is performed at a pH of between about 3 and about 10, between about 4 and about 9, between about 5 and about 8, and/or between about 6 and about 7. In embodiments, the reaction is performed at a pH of between about 5 and about 8.

In embodiments, the reaction at step 105 of method 100 is performed within a vessel designed to aid in generating crystals large enough for effective separation, efficient washing, and in robust scale up robust scaleup of macromixing (bulk convection), meso-mixing (turbulent eddies) and micro-mixing (including molecular and momentum diffusion related). In embodiments, the vessel may be a spinner flask, a conical tank, and the like.

In embodiments, the reaction composition comprises a nucleotide cofactor that can be converted to an NDP-sugar (e.g., NDP-glucose) by sucrose synthase. In some embodiments, the nucleotide can be ADP, GDP, UDP, CDP, or TDP. In some embodiments, the nucleotide can be a non-UDP nucleotide (i.e., ADP, GDP, CDP, or TDP). In another embodiment, the nucleotide is ADP. In a particular embodiment, the reaction can be carried out with e.g., ADP at a concentration between 0.01 mM and 10 mM, such as, for example, between 0.01 mM and 0.05 mM, between 0.05 mM and 0.1 mM, between 0.1 mM and 0.5 mM, between 0.5 mM and 1 mM, between 1 mM and 5 mM, or between 5 mM and 10 mM. In a particular embodiment, ADP is used at a concentration of 0.5 mM.

In embodiments, wherein the NDP-sugar or the sugar-phosphate is regenerated by transfer of a glycosyl moiety from a sugar dimer or oligomer. In embodiments, the sugar dimer is sucrose or maltose.

In embodiments, the reaction composition comprises sucrose and sucrose synthase, the sucrose can be present at a sucrose concentration between about 100 mM and about 3 M, about 125 mM and about 2.5 M, about 150 mM and about 2 M, about 175 mM and about 1.5 M, about 200 mM and about 1 M, about 225 mM and about 500 mM, or about 250 mM and about 300 mM. In an example, the reaction can be carried out with a sucrose concentration of 292 mM.

In embodiments, the reaction at step 105 of method 100 can be monitored by suitable methods including, but not limited to, high performance liquid chromatography (HPLC), liquid chromatography-mass spectrometry (LCMS), thin layer chromatography (TEC), infrared spectrometry (IR), or nuclear magnetic resonance (NMR).

After ERX is performed at step 105, the crystals are isolated at step 106 of method 100 by separation and/or washing. In embodiments, the result of ERX comprises crystallized steviol glycosides and a “mother liquor”, which comprises the buffer and any steviol glycosides that remain soluble. In embodiments, the precipitated steviol glycosides can be separated from the mother liquor by appropriate means including but not limited to vacuum filtration, centrifugation, and chamber filter press. In embodiments, the precipitated steviol glycosides can be further processed to enhance the purity of the precipitated steviol glycosides. The precipitated steviol glycosides can be washed with water to remove hydrophilic reaction components, such as residual sugars and salts. In embodiments, the precipitated steviol glycosides can be washed with an aqueous alcohol solvent to remove impurities. In embodiments, recovered and processed precipitated steviol glycosides can be further dried to form a powder. The precipitated steviol glycosides can be dried by any standard method including but not limited to freeze-drying, vacuum tray drying, spray drying, fluid-bed drying, filter mat drying, or rotary drum drying. In some embodiments, the dried ERX-crystals can be milled to generate a desired particle size.

The composition of the resulting crystallized steviol glycosides comprises one or more of the steviol glycosides described herein, including stevioside, Reb B, Reb G, Reb C, Reb F. Reb A, Reb I, Reb E, Reb E2, Reb AM, Reb H, Reb L, Reb K, Reb J, Reb M, Reb D, Reb N, Reb O, Reb Q, and synthetic steviol glycosides. The crystals may comprise, in an example, Reb D, Reb E, and Reb A. Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E, and Reb A may comprise between about 70% and about 100%, between about 72% and about 98%, between about 74% and about 96%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

In embodiments, the mother liquor can be further processed at sub process 110 of method 100. Outputs from sub process 110, which will be described in more detail below, can be optionally provided as recycle stream inputs to the starting composition.

Each of the isolation at step 106 and the processing of the mother liquor at sub process 110 generate products. In particular, ERX-crystals are generated by step 106. The types of ERX-crystals produced are based on the starting composition and the at least one enzyme used. In embodiments, the ERX-crystals may define a total suspended solids mass of the reaction, which may be between about 5% and about 50%, between about 10% and about 40%, and between about 15% and about 30%.

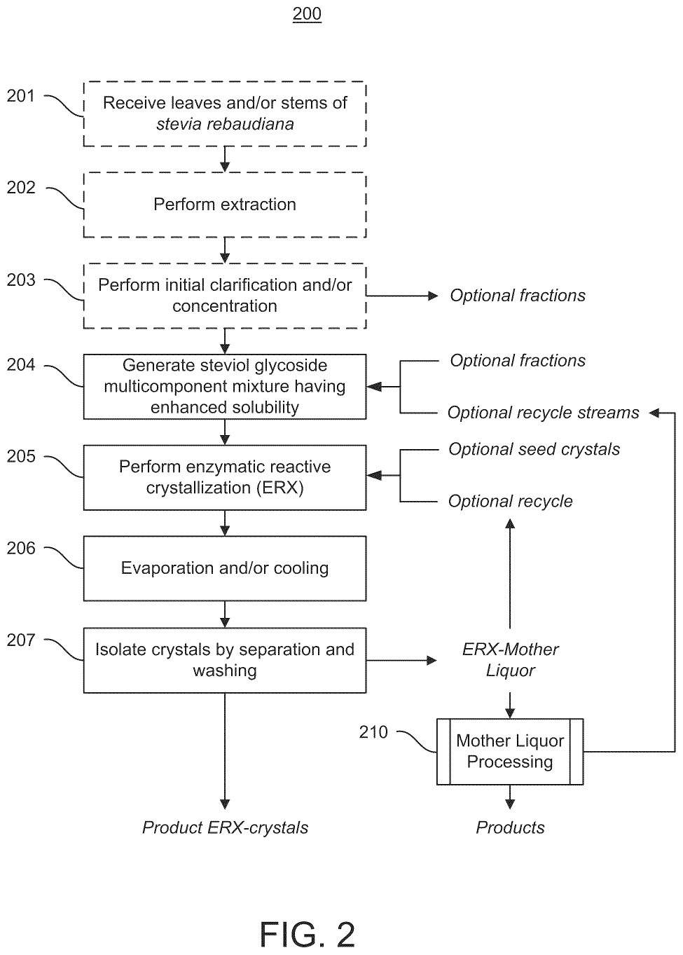

A variation of method 100 will now be described with reference to method 200 of FIG. 2. For brevity, certain repetitive descriptions will be omitted in favor of the corresponding descriptions above. At optional step 201 of method 200, leaves and/or stems of the Stevia rebaudiana may be received. At optional steps 202 and 203 of method 200, the leaves and/or stems of the Stevia rebaudiana may be processed to extract steviol glycosides and to perform initial clarification and/or concentration thereof. Fractions may be obtained during this processing and may be reintroduced at step 205, as shown in FIG. 2.

At step 204 of method 200, a starting composition, or steviol glycoside mixture having enhanced solubility, can be generated from the processed Stevia leaf extract. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 205 of method 200, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme is added to the reaction composition at the start of the reaction. For instance, when the at least one enzyme comprises a β12GT and a β13GT, the glycosyltransferase may be provided together at the start of the reaction. In another embodiment, a first portion of the at least one enzyme can be added at the start of the reaction and one or more additional portions of the at least one enzyme can be added during the time course of the reaction. For example, timing can be adjusted in view of expected conversion products and in view of enzyme activity. In embodiments, the aqueous reaction mixture comprises one or more of sodium chloride, sodium acetate, potassium chloride, sodium sulfate, and sodium phosphate to aid in maintaining enzyme solubility.

In embodiments, the at least one enzyme can be provided in any suitable form, including free, immobilized, or as a whole cell system. The degree of purity of the at least one enzyme may vary. For example, it may be provided as a crude, semi-purified, or purified enzyme preparation(s). In one embodiment, the at least one enzyme is free. In another embodiment, the at least one enzyme is immobilized to a solid support, for example on an inorganic or organic support. In some embodiments, the solid support is derivatized cellulose, glass, ceramic, methacrylate, styrene, acrylic, a metal oxide, or a membrane. In some embodiments, the at least one enzyme is immobilized to the solid support by covalent attachment, adsorption, cross-linking, entrapment, or encapsulation. In some embodiments, the at least one enzyme is provided in the form of a whole cell system, for example as a living fermentative microbial cell, or as dead and stabilized microbial cell, or in the form of a cell lysate.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides.

FIG. 13 is a flow diagram illustrating the role of each of B12GT and B13GT within a stevioside to Reb M pathway. In embodiments, the B12GT may be add a beta-linked glucose monomer to the C2′ of the 13-O-glucose and/or 19-O-glucose of a steviol glycoside substrate. For example, the B13GT may use an ADP-glucose sugar donor convert Stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may add a beta-linked glucose monomer to the C3′ of the 13-O-glucose and/or 19-O-glucose of a steviol glycoside substrate. For example, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

In embodiments, the at least one enzyme comprises one or more sucrose synthases. The one or more sucrose synthases may be a wild type sucrose synthase, a non-natural, engineered sucrose synthase, a polypeptide having sucrose synthase activity, or a combination thereof. In embodiments, the one or more sucrose synthases (SuSy) may use a sucrose sugar donor to convert an NDP and sucrose to form NDP-glucose and fructose. For example, the one or more SuSy may convert ADP and sucrose to ADP-glucose and fructose. In embodiments, the SuSy may be a non-natural, engineered SuSy as described in International Patent Application No. PCT/US2022/016820, which is incorporated herein by reference in its entirety.

In variations, seed crystals may optionally be added to the reaction at step 205 of method 200. Seed crystals can be added to the reaction to promote crystallization/precipitation of the crystallized steviol glycosides. The seed crystals can be generated enzymatically by reacting glycosyltransferases with a mixture of steviol glycosides. For example, an initial enzyme dosage of less than 1/10 a final enzyme dosage can be reacted with the starting composition to begin crystal formation. Such initial crystal formation can be performed for at least 2 minutes, at least 3 minutes, at least 4 minutes, at least 5 minutes, at least 10 minutes, at least 20 minutes, at least 30 minutes, at least 40 minutes, at least 50 minutes, and/or at least 60 minutes.

After ERX is performed at step 205, the reacted composition can be further processed at step 206 of method 200 by evaporation and/or cooling and/or other technique for promoting and enhancing crystal formation. Evaporation may be referred to as evaporation crystallization. Cooling may be referred to as cooling crystallization and may comprise cooling via heat transfer surface cooling, evaporative cooling, or a combination thereof. In embodiments, the cooling includes significant evaporation and reducing the temperature to less than or equal to that of the ERX reaction temperature, thereby recovering the product crystals.

Subsequently, the crystals can be isolated at step 206 of method 200 by separation and/or washing. In embodiments, the result of ERX comprises crystallized steviol glycosides and a “mother liquor”, which comprises the buffer and any steviol glycosides that remain soluble. In embodiments, the mother liquor can be provided as a recycle stream to ERX at step 205 of method 200 and/or further processed at sub process 210 of method 200. Outputs from sub process 210, which will be described in more detail below, can be optionally provided as recycle stream inputs to the starting composition.

Each of the isolation at step 206 and the processing of the mother liquor at sub process 210 generate products. In particular, ERX-crystals are generated by step 206. The types of ERX-crystals produced are based on the starting composition and the at least one enzyme used.

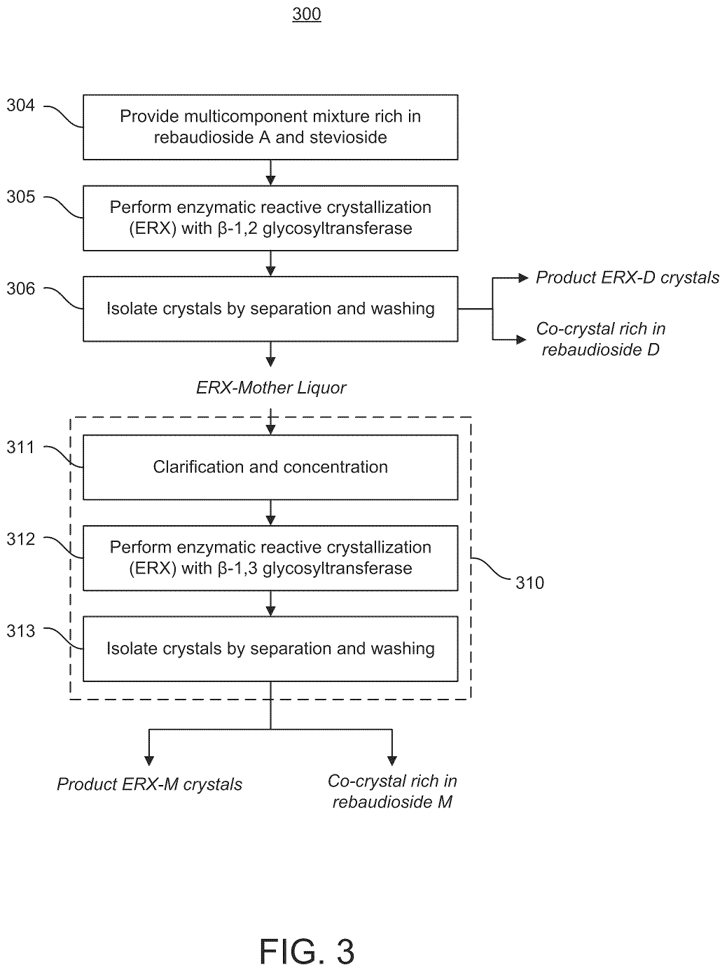

A variation of method 100 will now be described with reference to method 300 of FIG. 3, wherein products include Red D crystals (“ERX-D crystals”), co-crystals rich in Reb D, Reb M crystals (“ERX-M crystals”), and co-crystals rich in rebaudioside M. For brevity, certain repetitive descriptions will be omitted in favor of the corresponding descriptions above.

In embodiments, the mother liquor can be further processed at sub process 310 of method 300. Sub process 310 is substantially similar to sub process 110, 210, and other variations of mother liquor processing described herein. To this end, the soluble steviol glycosides from the mother liquor can be purified to generate another steviol glycoside product. For example, a reactive crystallization that reacts a B12GT with an RA50-based starting composition can generate reaction solids primarily comprising Reb D and a soluble mother liquor primarily comprising Reb E. The soluble mother liquor can then be subsequently processed to generate a Reb E product. In embodiments, the soluble reaction mother liquor, which may comprise solubilized, un-crystallized Reb D, can be used as a starting composition for a new enzymatic reaction and/or ERX reaction.

At step 311 of sub process 310, mother liquor containing Reb E and solubilized, un-crystallized Reb D, can be clarified and concentrated.

At step 312 of sub process 310, the mother liquor can then be reacted with a reaction composition to perform ERX (or a second ERX). In embodiments, the reaction composition comprises at least one enzyme. For example, the at least one enzyme may comprise B13GT. The B13GT may be a wild type glycosyltransferase, a non-natural, engineered glycosyltransferase, a polypeptide having glycosyltransferase activity, or a combination thereof. In embodiments, the B13GT may be a non-natural, engineered B13GT glycosyltransferase as described in International Patent Application No. PCT/US2023/073344, which is incorporated herein by reference in its entirety.

After ERX is performed at step 312, the reacted composition can be further processed at step 312 of sub process 310 by separation and/or washing to isolate crystallized Reb M. In embodiments, the result of ERX at step 312 comprises crystallized steviol glycosides and a mother liquor. After isolation at step 313 of sub process 310, Reb M crystals and co-crystals rich in Reb M are generated.

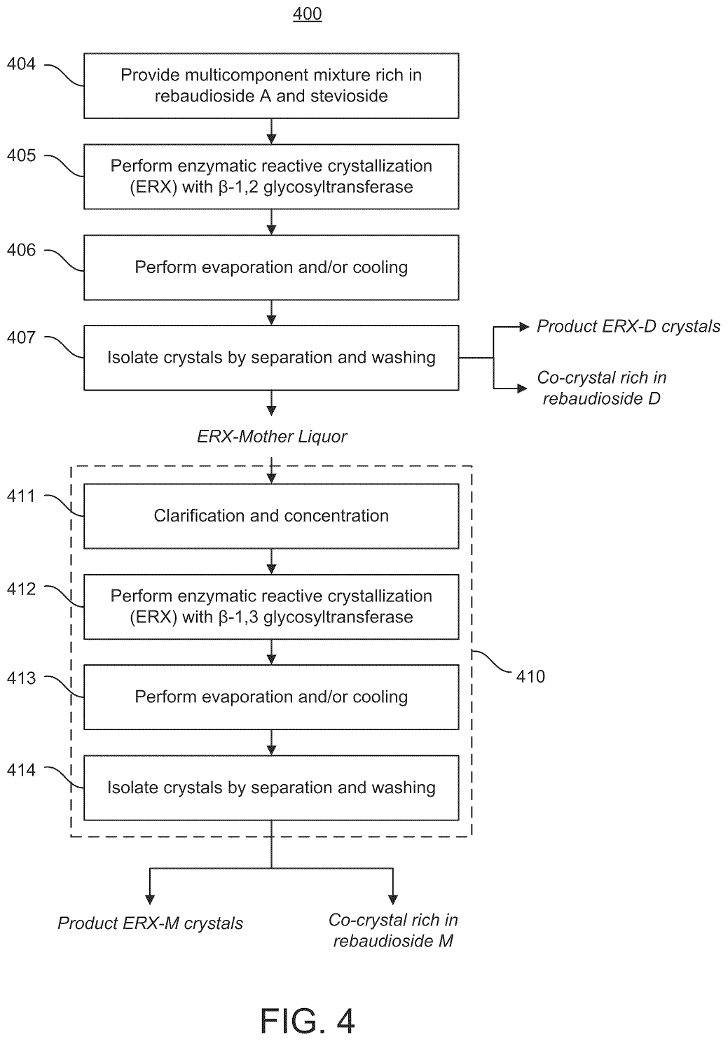

A variation of method 300 will now be described with reference to method 400 of FIG. 4, wherein products include Red D crystals (“ERX-D crystals”), co-crystals rich in Reb D, Reb M crystals (“ERX-M crystals”), and co-crystals rich in rebaudioside M. For brevity, certain repetitive descriptions will be omitted in favor of the corresponding descriptions above.

At step 404 of method 400, a starting composition, or steviol glycoside mixture having enhanced solubility, can be provided. The starting composition can comprise Stevia leaf extract as described above. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 405 of method 400, the starting composition can be reacted with a reaction composition to perform enzymatic reactive crystallization (ERX). In embodiments, the reaction composition comprises at least one enzyme. For example, the at least one enzyme may comprise B12GT. The B12GT may be a wild type glycosyltransferase, a non-natural, engineered glycosyltransferase, a polypeptide having glycosyltransferase activity, or a combination thereof. In embodiments, the B12GT may be a non-natural, engineered B12GT glycosyltransferase as described in International Patent Application No. PCT/US2022/016820, which is incorporated herein by reference in its entirety.

After ERX is performed at step 405, the reacted composition can be further processed. At step 406 of method 400, evaporation and/or cooling and/or other technique for promoting and enhancing crystal formation can be implemented. Subsequently, at step 407 of method 400, the reaction solids, or crystals, can be isolated by separation and/or washing. In embodiments, the result of ERX comprises crystallized steviol glycosides and a “mother liquor”, which comprises the buffer and any steviol glycosides that remain soluble. As shown in FIG. 4, the isolated crystals from step 407 of method 400 comprise Reb D crystals and co-crystals rich in Reb D. In embodiments, the co-crystals rich in Reb D may comprise at least about 50% Reb D, about 55% Reb D, about 60% Reb D, about 65% Reb D, about 70% Reb D, about 75% Reb D, about 80% Reb D, about 85% Reb D, about 90% Reb D, about 91% Reb D, about 92% Reb D, about 93% Reb D, about 94% Reb D, about 95% Reb D, about %% Reb D, about 97% Reb D, about 98% Reb D, or about 99% Reb D.

In embodiments, the mother liquor can be further processed at sub process 410 of method 400. Sub process 410 is substantially similar to sub process 110, 210, 310, and other variations of mother liquor processing described herein. To this end, the soluble steviol glycosides from the mother liquor can be purified to generate another steviol glycoside product. For example, a reactive crystallization that reacts a B12GT with an RA50-based starting composition can generate reaction solids primarily comprising Reb D and a soluble mother liquor primarily comprising Reb E. The soluble mother liquor can then be subsequently processed to generate a Reb E product. In embodiments, the soluble reaction mother liquor, which may comprise solubilized, un-crystallized Reb D, can be used as a starting composition for a new enzymatic reaction and/or ERX reaction.

At step 411 of sub process 410, mother liquor containing Reb E and solubilized, un-crystallized Reb D, can be clarified and concentrated.

At step 412 of sub process 410, the mother liquor can then be reacted with a reaction composition to perform ERX. In embodiments, the reaction composition comprises at least one enzyme. For example, the at least one enzyme may comprise B13GT. The B13GT may be a wild type glycosyltransferase, a non-natural, engineered glycosyltransferase, a polypeptide having glycosyltransferase activity, or a combination thereof. In embodiments, the B13GT may be a non-natural, engineered B13GT glycosyltransferase as described in International Patent Application No. PCT/US2023/073344, which is incorporated herein by reference in its entirety.

After ERX is performed at step 412, the reacted composition can be further processed at step 413 of sub process 410 by evaporation and/or cooling and/or other technique for promoting and enhancing crystal formation. Subsequently, crystals can be isolated at step 414 of sub process 410 by separation and/or washing to isolate crystallized Reb M. In embodiments, the result of ERX at step 412 comprises crystallized steviol glycosides and a mother liquor. After isolation at step 414 of sub process 410, Reb M crystals and co-crystals rich in Reb M are generated. In embodiments, the co-crystals rich in Reb M may comprise at least about 50% Reb M, about 55% Reb M, about 60% Reb M, about 65% Reb M, about 70% Reb M, about 75% Reb M, about 80% Reb M, about 85% Reb M, about 90% Reb M, about 91% Reb M, about 92% Reb M, about 93% Reb M, about 94% Reb M, about 95% Reb M, about 96% Reb M, about 97% Reb M, about 98% Reb M, or about 99% Reb M.

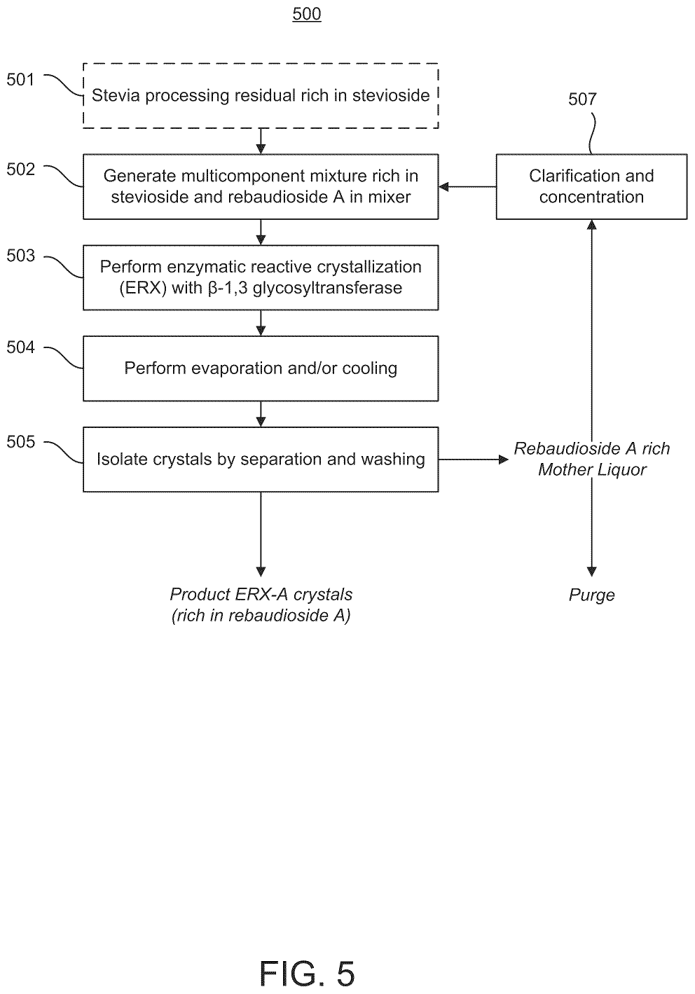

A variation of the above methods will now be described with reference to method 500 of FIG. 5, wherein products include Red A crystals (“ERX-A crystals”). For brevity, certain repetitive descriptions will be omitted in favor of the corresponding descriptions above.

After obtaining a Stevia processing residual rich in stevioside (e.g., the residual from processing Stevia leaf to obtain a Reb A rich stream) from step 501 of method 500, a starting composition, or steviol glycoside mixture having enhanced solubility, can be prepared within a mixer at step 502 of method 500. The mixer may be a dissolver mixer and the like. For example, the mixer may a rotor-stator high shear blender. The starting composition of may also comprise clarified and concentrated Reb A rich mother liquor derived at step 507 of method 500 (assuming a Reb A rich mother liquor is previously available). In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50.

At step 503 of method 500, the starting composition can be reacted with a reaction composition to perform enzymatic reactive crystallization (ERX). In embodiments, the reaction composition comprises at least one enzyme. For example, the at least one enzyme may comprise B13GT. The B13GT may be a wild type glycosyltransferase, a non-natural, engineered glycosyltransferase, a polypeptide having glycosyltransferase activity, or a combination thereof. In embodiments, the B13GT may be a non-natural, engineered B13GT glycosyltransferase as described in International Patent Application No. PCT/US2023/073344, which is incorporated herein by reference in its entirety.

After ERX is performed at step 503, the reacted composition can be further processed. At step 504 of method 500, evaporation and/or cooling and/or other technique for promoting and enhancing crystal formation can be implemented. Subsequently, at step 505 of method 500, the reaction solids, or crystals, can be isolated by separation and/or washing. In embodiments, the result of ERX comprises crystallized steviol glycosides and a “mother liquor”, which comprises the buffer and any steviol glycosides that remain soluble. As shown in FIG. 5, the isolated crystals from step 505 of method 500 comprise Reb A crystals and the mother liquor is rich in soluble Reb A. In embodiments, the co-crystals rich in Reb A may comprise at least about 50% Reb A, about 55% Reb A, about 60% Reb A, about 65% Reb A, about 70% Reb A, about 75% Reb A, about 80% Reb A, about 85% Reb A, about 90% Reb A, about 91% Reb A, about 92% Reb A, about 93% Reb A, about 94% Reb A, about 95% Reb A, about 96% Reb A, about 97% Reb A, about 98% Reb A, or about 99% Reb A.

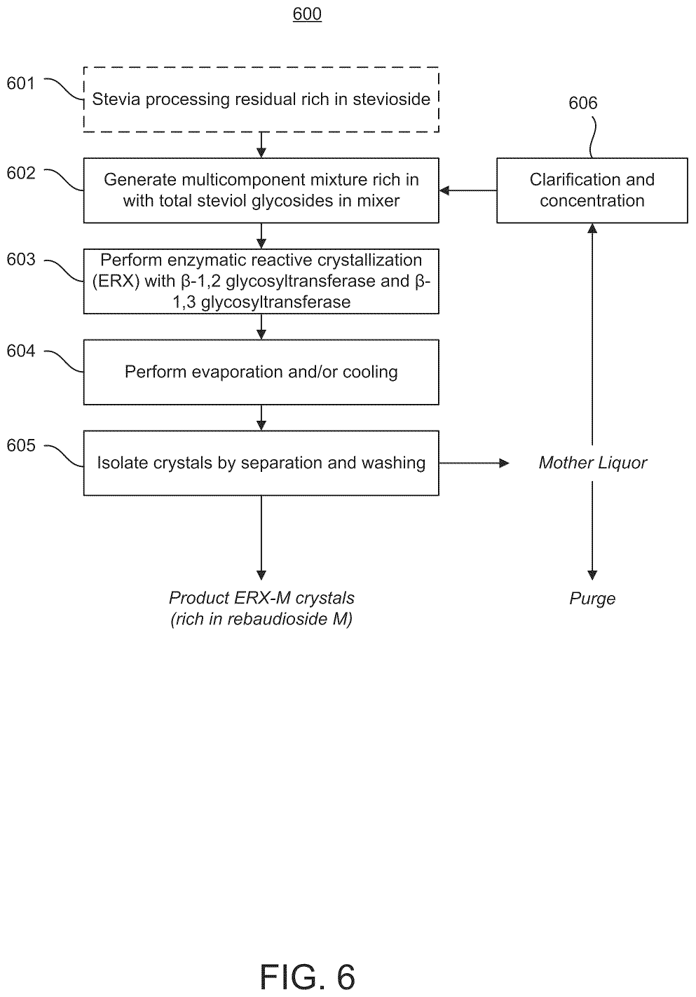

A variation of the method 500 will now be described with reference to method 600 of FIG. 6, wherein products include Red M crystals (“ERX-M crystals”). For brevity, certain repetitive descriptions will be omitted in favor of the corresponding descriptions above.

After obtaining a Stevia processing residual rich in stevioside (e.g., the residual from processing Stevia leaf to obtain a Reb A rich stream) from step 601 of method 600, a starting composition, or steviol glycoside mixture having enhanced solubility, can be prepared within a mixer at step 602 of method 600. The mixer may be a dissolver mixer and the like. The starting composition of may also comprise clarified and concentrated mother liquor derived at step 607 of method 600. The mother liquor may comprise soluble forms of one or more of the steviol glycosides described herein.

At step 603 of method 600, the starting composition can be reacted with a reaction composition to perform enzymatic reactive crystallization (ERX). In embodiments, the reaction composition comprises at least one enzyme. For example, the at least one enzyme may comprise B12GT and B13GT. The B12GT may be a wild type glycosyltransferase, a non-natural, engineered glycosyltransferase, a polypeptide having glycosyltransferase activity, or a combination thereof. In embodiments, the B12GT may be a non-natural, engineered B12GT glycosyltransferase as described in International Patent Application No. PCT/US2022/016820, which is incorporated herein by reference in its entirety. The B13GT may be a wild type glycosyltransferase, a non-natural, engineered glycosyltransferase, a polypeptide having glycosyltransferase activity, or a combination thereof. In embodiments, the B13GT may be a non-natural, engineered B13GT glycosyltransferase as described in International Patent Application No. PCT/US2023/073344, which is incorporated herein by reference in its entirety.

After ERX is performed at step 603, the reacted composition can be further processed. At step 604 of method 600, evaporation and/or cooling and/or other technique for promoting and enhancing crystal formation can be implemented. Subsequently, at step 605 of method 600, the reaction solids, or crystals, can be isolated by separation and/or washing. In embodiments, the result of ERX comprises crystallized steviol glycosides and a “mother liquor”, which comprises the buffer and any steviol glycosides that remain soluble. As shown in FIG. 6, the isolated crystals from step 605 of method 600 comprise Reb M crystals and the mother liquor is rich in soluble steviol glycosides.

As shown in each of FIG. 5 and FIG. 6, the soluble ERX mother liquor can be recycled to improve the solubility of a steviol glycoside starting composition at step 502 of method 500 and at step 602 of method 600. For example, as shown in FIG. 5, a starting composition of stevioside, when reacted with a B13GT, will produce a soluble mother liquor rich in Reb A. The Reb A can be recycled and mixed with the stevioside rich starting composition to create an enhanced solubility starting composition.

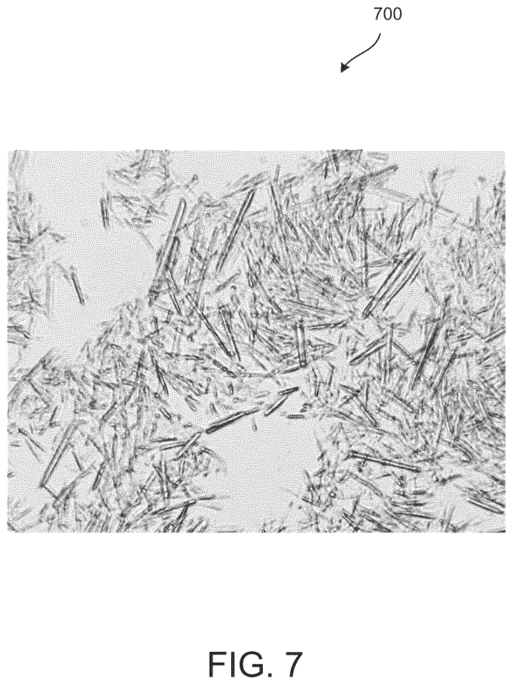

FIG. 7 is an image of rebaudioside D-rich co-crystals produced by the ERX methods described herein. The ERX method deploys RA50 at 15-liter reactor scale, vacuum oven drying, suspension in water, and light microscopy at 400× magnification.

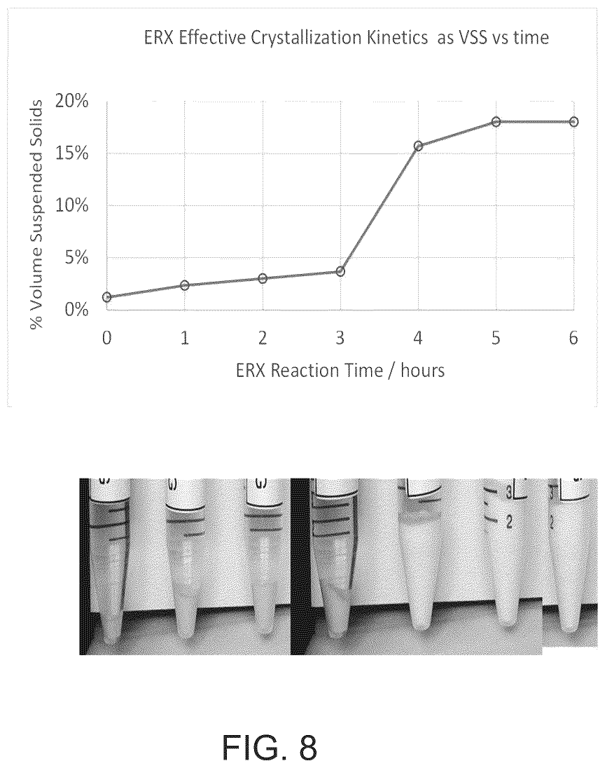

FIG. 8 depicts crystallization kinetics of an ERX method as measured by volume percent suspended solids present in a 150-liter scale ERX run.

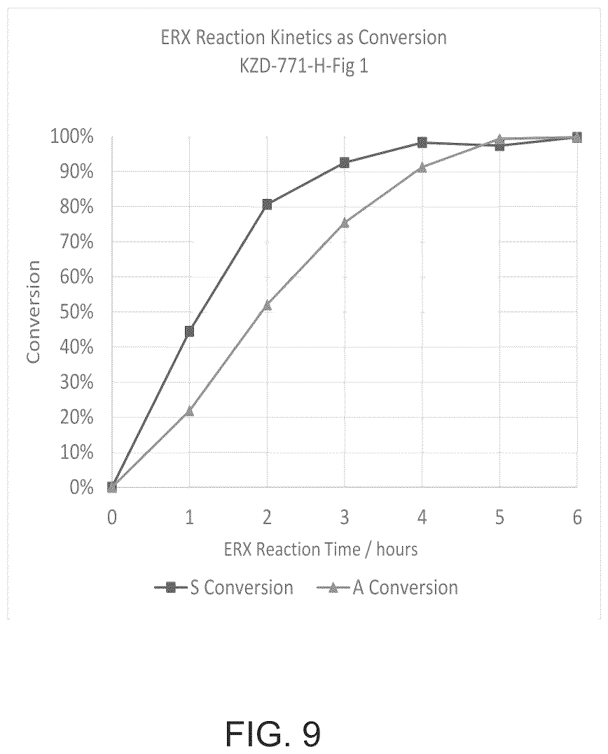

FIG. 9 is a graphical illustration of reaction kinetics of an ERX method as measured by conversion of stevioside and conversion of rebaudioside A in a 150-liter scale ERX run.

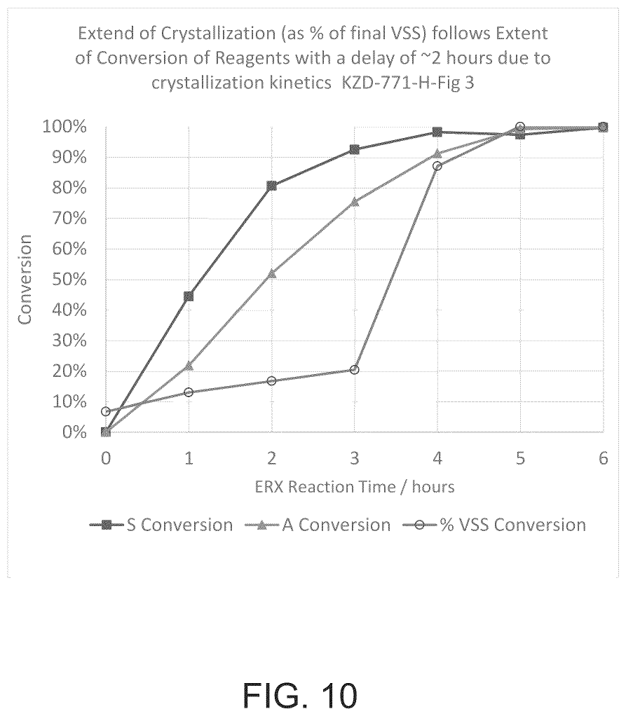

FIG. 10 is a graphical illustration of an overlay of the crystallization kinetics of FIG. 8 and the reaction kinetics from FIG. 9, illustrating a time-lag or time-offset between reaction and crystal formation.

Together, FIG. 9 and FIG. 10 illustrate how the rate of conversion of stevioside and Reb A to Reb D or Reb M aligns with the formation of crystallized product. Such data can be useful for determining enzyme dosing and other reaction parameters to realize conversion of the feedstock components to the final product in solution and then the precipitated solids.

FIG. 11 is a graphical illustration of a theoretical calculation of sucrose equivalent, per kilogram of harvested leaf, showing substantial value in effective sweetness obtainable by enzymatically upgrading the leaf extract. Steviol glycosides containing rhamnosyl moieties, which are excluded from this graphic for visual clarity, have significantly lower sucrose equivalence.

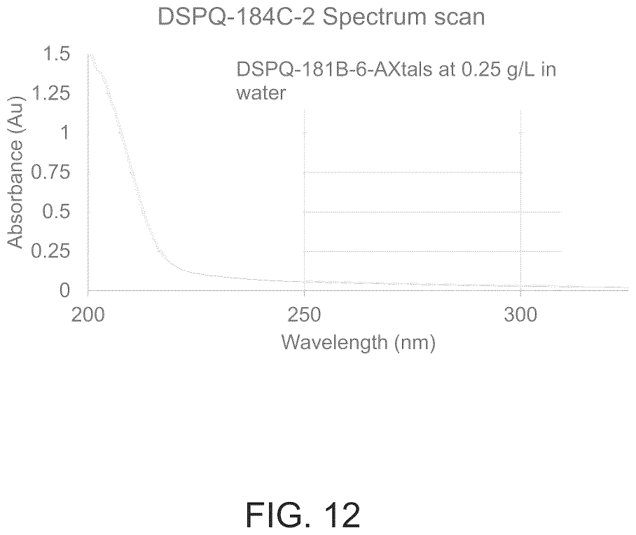

FIG. 12 is a UV-Vis spectrophotometric scan of crystals produced at laboratory scale by an ERX method, showing excellent purity in regard to absence of impurities that adsorb at 254 nm, 270 nm, 280 nm, and 315 nm.

FIG. 13 shows the enzymatic reactions carried out by a beta-1,2-glycosyltransferase and a beta-1,3-glycosyltransferase to convert stevioside to rebaudioside M.

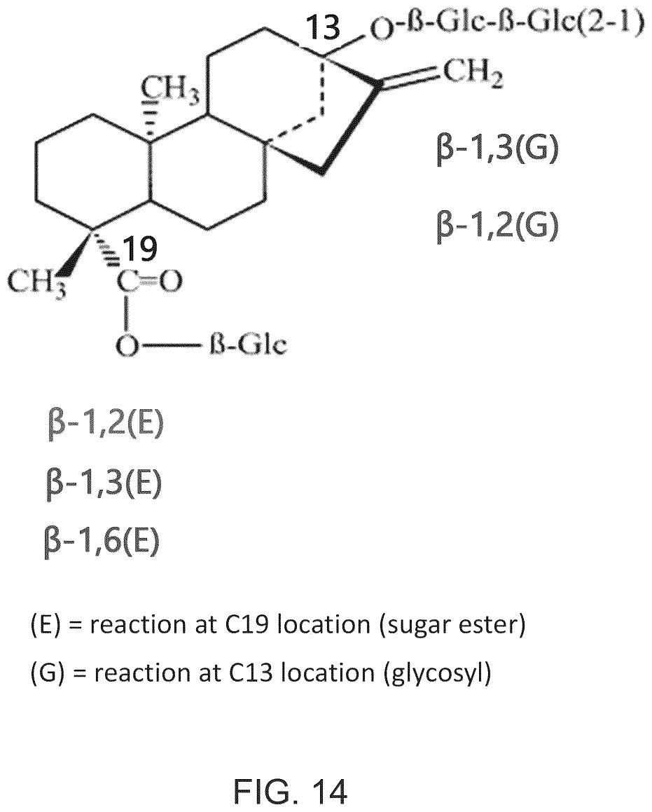

FIG. 14 shows the stevioside molecule and indicates to which carbon (C13, (G) reaction, or C19 (E) reaction) a beta-1,2-glycosyltransferase and a beta-1,3-glycosyltransferase can attach additional glucose units during the conversion to rebaudioside M.

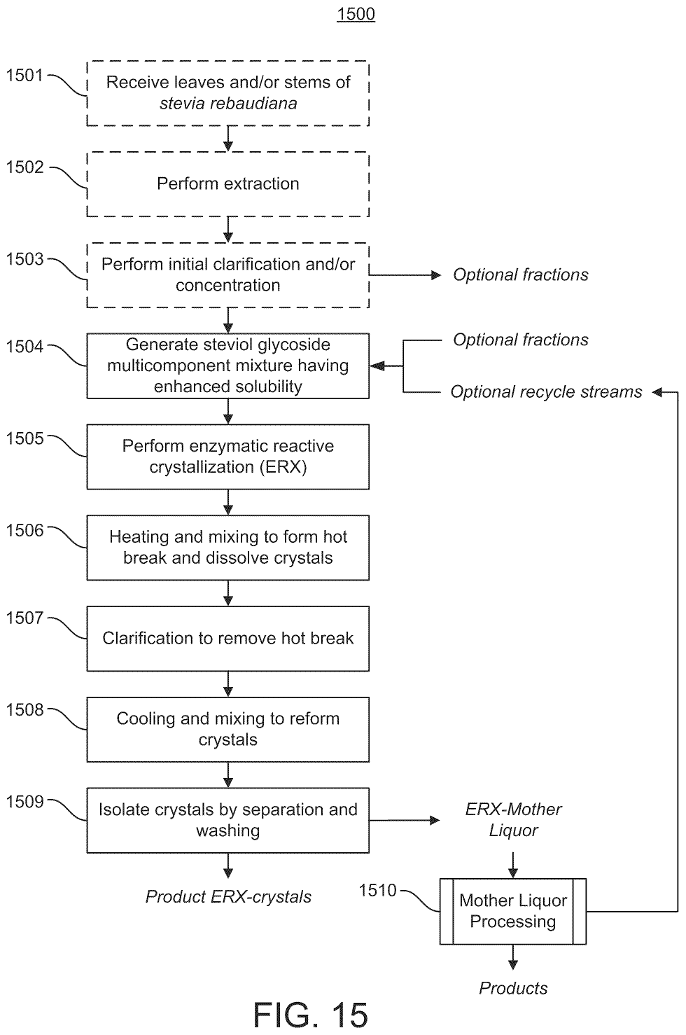

Referring now to FIG. 15, a method of generating and isolating steviol glycoside crystals will be described with reference to method 1500.

At optional step 1501 of method 1500, leaves and/or stems of the Stevia rebaudiana may be received. At optional steps 1502 and 1503 of method 1500, the leaves and/or stems of the Stevia rebaudiana may be processed to extract steviol glycosides and to perform initial clarification and/or concentration thereof. Fractions may be obtained during this processing and may be reintroduced at step 1505, as shown in FIG. 15.

At step 1504 of method 1500, a starting composition, or steviol glycoside mixture having enhanced solubility, can be generated from the processed Stevia leaf extract or otherwise provided. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 1505 of method 1500, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides. For example, a B12GT may use an ADP-glucose sugar donor to convert stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

After ERX is performed at step 1505, the ERX reaction product can be processed to enhance the steviol glycoside purity prior to separation of the ERX reaction product crystals. As shown at step 1506 of method 1500, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C., and about 40° C. between about 40° C., and about 50° C. between about 50° C., and about 60° C., between about 60° C., and about 70° C., between about 70° C. and about 80° C., between about 80° C., and about 90° C., between about 90° C., and about 100° C. between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1506 of method 1500 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

At step 1507 of method 1500, a clarification can be performed to remove the hot break (i.e., remove impurities). In embodiments, clarifying comprises centrifugation, filtration, or a combination thereof. In embodiments, the hot break comprises proteins and other cellular derived debris. In embodiments, the clarification can comprise removing a solvent break.

Crystal reformation can be performed via cooling crystallization at step 1508 of method 1500. In embodiments, the cooling crystallization comprises cooling via heat transfer surface cooling, evaporative cooling, or a combination thereof. In embodiments, the cooling includes significant evaporation and reducing the temperature to less than or equal to that of the ERX reaction temperature, thereby recovering the product crystals.

The reformed crystals can then be isolated at step 1509 of method 1500 by separation and/or washing. In embodiments, the result of ERX, reheating, and recrystallization comprises recrystallized steviol glycosides and a “mother liquor”, which comprises the buffer and any steviol glycosides that remain soluble. In embodiments, the precipitated steviol glycosides can be separated from the mother liquor by appropriate means including but not limited to vacuum filtration, centrifugation, and chamber filter press. In embodiments, the precipitated steviol glycosides can be further processed to enhance the purity of the precipitated steviol glycosides. The precipitated steviol glycosides can be washed with water to remove hydrophilic reaction components, such as residual sugars and salts. In embodiments, the precipitated steviol glycosides can be washed with an aqueous alcohol solvent to remove impurities. In embodiments, recovered and processed precipitated steviol glycosides can be further dried to form a powder. The precipitated steviol glycosides can be dried by any standard method including but not limited to freeze-drying, vacuum tray drying, spray drying, fluid-bed drying, filter mat drying, or rotary drum drying. In some embodiments, the dried ERX-crystals can be milled to generate a desired particle size.

The composition of the resulting crystallized steviol glycosides comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The crystals may comprise, in an example, Reb D, Reb E, and Reb A. Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E, and Reb A may comprise between about 70% and about 100%, between about 72% and about 98%, between about 74% and about %%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

In embodiments, the mother liquor can be further processed at sub process 1510 of method 1500. Outputs from sub process 1510, which are described elsewhere herein, can be optionally provided as recycle stream inputs to the starting composition.

Each of the isolation at step 1506 and the processing of the mother liquor at sub process 1510 generate products. In particular, ERX-crystals are generated by step 1506. The types of ERX-crystals produced are based on the starting composition and the at least one enzyme used.

In some embodiments, steps 1506-1507 of method 1500 comprise mixing the ERX crystals with a solvent, heating the slurry to a temperature such that ERX crystals substantially dissolve, clarifying to remove any particulate or insoluble materials, and/or cooling the slurry to the ERX reaction temperature to form crystals. In some embodiments, the heating temperature may be greater than the ERX reaction temperature. For instance, the heating temperature may be greater than about 92° C. In embodiments, the heating temperature may be greater than about 78° C., and/or greater than about 110° C., and/or may be applied under pressure. In embodiments, the heating temperature may be greater than about 92° C., greater than about 110° C., and/or greater than about 130° C., and/or may be applied under pressure. In embodiments, the solvent may be water, alcohol, methanol, ethanol, isopropanol, n-propanol, or isoamyl alcohol.

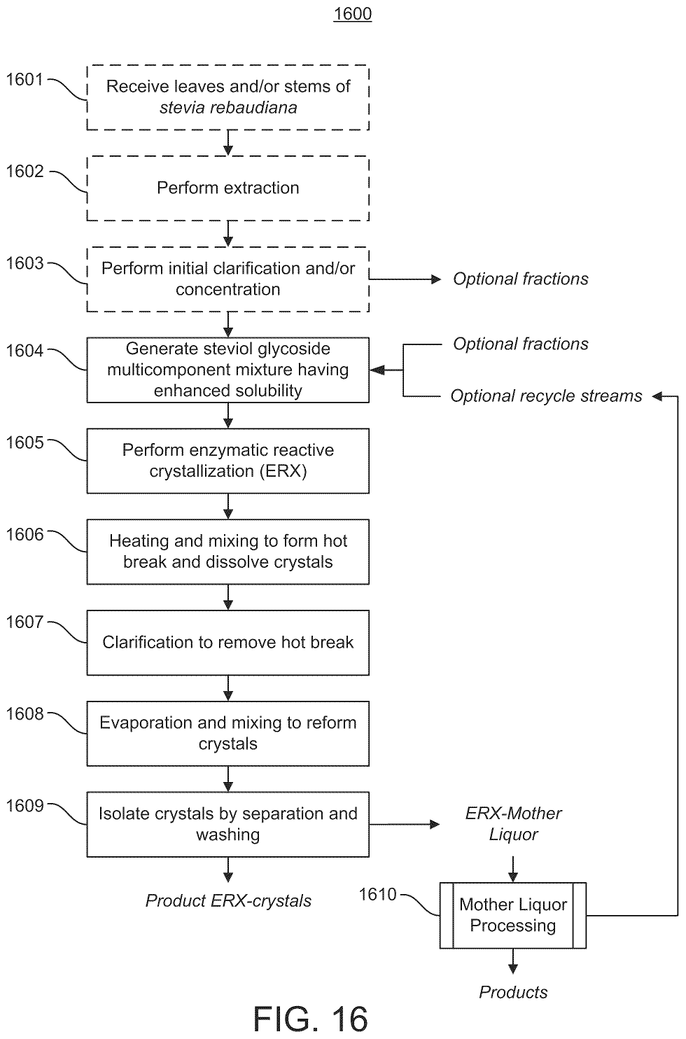

Referring now to FIG. 16, a method of generating and isolating steviol glycoside crystals will be described with reference to method 1600.

At optional step 1601 of method 1600, leaves and/or stems of the Stevia rebaudiana may be received. At optional steps 1602 and 1603 of method 1600, the leaves and/or stems of the Stevia rebaudiana may be processed to extract steviol glycosides and to perform initial clarification and/or concentration thereof. Fractions may be obtained during this processing and may be reintroduced at step 1605, as shown in FIG. 16.

At step 1604 of method 1600, a starting composition, or steviol glycoside mixture having enhanced solubility, can be generated from the processed Stevia leaf extract or otherwise provided. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 1605 of method 1600, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides. For example, a B12GT may use an ADP-glucose sugar donor to convert stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

After ERX is performed at step 1605, the ERX reaction product can be processed to enhance the steviol glycoside purity prior to separation of the ERX reaction product crystals. As shown at step 1606 of method 1600, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C., and about 40° C., between about 40° C., and about 50° C., between about 50° C., and about 60° C., between about 60° C., and about 70° C., between about 70° C. and about 80° C., between about 80° C., and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1506 of method 1500 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

At step 1607 of method 1600, a clarification can be performed to remove the hot break (i.e., remove impurities). In embodiments, clarifying comprises centrifugation, filtration, or a combination thereof. In embodiments, the hot break comprises proteins and other cellular derived debris. In embodiments, the clarification can comprise removing a solvent break. Crystal reformation can be performed via evaporation crystallization at step 1608 of method 1600. The reformed crystals can then be isolated at step 1609 of method 1600 by separation and/or washing. In embodiments, the result of ERX, reheating, and recrystallization comprises recrystallized steviol glycosides and a “mother liquor”, which comprises the buffer and any steviol glycosides that remain soluble. In embodiments, the precipitated steviol glycosides can be separated from the mother liquor by appropriate means including but not limited to vacuum filtration, centrifugation, and chamber filter press. In embodiments, the precipitated steviol glycosides can be further processed to enhance the purity of the precipitated steviol glycosides. The precipitated steviol glycosides can be washed with water to remove hydrophilic reaction components, such as residual sugars and salts. In embodiments, the precipitated steviol glycosides can be washed with an aqueous alcohol solvent to remove impurities. In embodiments, recovered and processed precipitated steviol glycosides can be further dried to form a powder. The precipitated steviol glycosides can be dried by any standard method including but not limited to freeze-drying, vacuum tray drying, spray drying, fluid-bed drying, filter mat drying, or rotary drum drying. In some embodiments, the dried ERX-crystals can be milled to generate a desired particle size.

The composition of the resulting crystallized steviol glycosides comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The crystals may comprise, in an example, Reb D, Reb E, and Reb A, Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E. and Reb A may comprise between about 70% and about 100%, between about 72% and about 98%, between about 74% and about 96%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb 1, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

In embodiments, the mother liquor can be further processed at sub process 1610 of method 1600. Outputs from sub process 1610, which are described elsewhere herein, can be optionally provided as recycle stream inputs to the starting composition.

Each of the isolation at step 1606 and the processing of the mother liquor at sub process 1610 generate products. In particular, ERX-crystals are generated by step 1606. The types of ERX-crystals produced are based on the starting composition and the at least one enzyme used.

In some embodiments, steps 1606-1607 of method 1600 comprise mixing the ERX crystals with a solvent, heating the mixture to a temperature such that ERX crystals substantially dissolve, clarifying to remove any particulate or insoluble materials, and/or cooling the mixture to the ERX reaction temperature to form crystals. In embodiments, the mixture may be heated to a temperature between about 10° C., and about 150° C., between about 10° C., and about 20° C., between about 20° C., and about 30° C., between about 30° C., and about 40° C., between about 40° C., and about 50° C., between about 50° C., and about 60° C., between about 60° C., and about 70° C., between about 70° C., and about 80° C., between about 80° C., and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In some embodiments, the heating temperature may be greater than the ERX reaction temperature. For instance, the heating temperature may be greater than about 92° C. In embodiments, the heating temperature may be greater than about 78° C., and/or greater than about 110° C., and/or may be applied under pressure. In embodiments, the heating temperature may be greater than about 92° C., greater than about 110° C., and/or greater than about 130° C., and/or may be applied under pressure. In embodiments, the solvent may be water, alcohol, methanol, ethanol, isopropanol, n-propanol, or isoamyl alcohol.

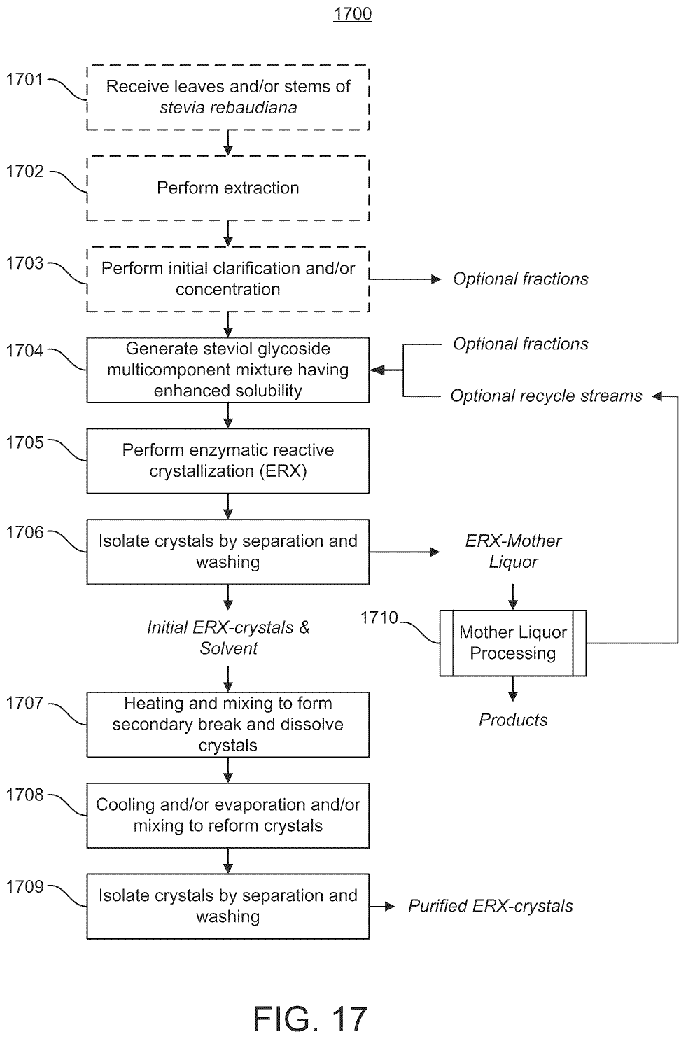

Referring now to FIG. 17, a method comprising further processing of the ERX reaction product to enhance steviol glycoside purity after separation of the ERX reaction product crystals is described. Certain steps of method 1700 of FIG. 17 are substantially similar to those of method 100 of FIG. 1, and so certain steps will be omitted or abbreviated for brevity.

At optional step 1701 of method 1700, leaves and/or stems of the Stevia rebaudiana may be received. At optional steps 1702 and 1703 of method 1700, the leaves and/or stems of the Stevia rebaudiana may be processed to extract steviol glycosides and to perform initial clarification and/or concentration thereof. Fractions may be obtained during this processing and may be reintroduced at step 1705, as shown in FIG. 17.

At step 1704 of method 1700, a starting composition, or steviol glycoside mixture having enhanced solubility, can be generated from the processed Stevia leaf extract. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50.

Alternatively, the starting composition may be obtained as an already processed version of a Stevia rebaudiana. Moreover, the starting composition can be synthetic or at least partially purified, commercially available, or otherwise prepared.

At step 1705 of method 1700, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides.

FIG. 13 is a flow diagram illustrating the role of each of B12GT and B13GT within a stevioside to Reb M pathway. In embodiments, the B12GT may be add a beta-linked glucose monomer to the C2′ of the 13-O-glucose and/or 19-O-glucose of a steviol glycoside substrate. For example, the B13GT may use an ADP-glucose sugar donor convert Stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may add a beta-linked glucose monomer to the C3′ of the 13-O-glucose and/or 19-O-glucose of a steviol glycoside substrate. For example, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

After ERX is performed at step 1705, the crystals are isolated at step 1706 of method 1700 by separation and/or washing. In embodiments, the result of ERX comprises crystallized steviol glycosides and a “mother liquor”, which comprises the buffer and any steviol glycosides that remain soluble. In embodiments, the precipitated steviol glycosides can be separated from the mother liquor by appropriate means including but not limited to vacuum filtration, centrifugation, and chamber filter press. In embodiments, the precipitated steviol glycosides can be further processed to enhance the purity of the precipitated steviol glycosides. The precipitated steviol glycosides can be washed with water to remove hydrophilic reaction components, such as residual sugars and salts. In embodiments, the precipitated steviol glycosides can be washed with an aqueous alcohol solvent to remove impurities. In embodiments, recovered and processed precipitated steviol glycosides can be further dried to form a powder. The precipitated steviol glycosides can be dried by any standard method including but not limited to freeze-drying, vacuum tray drying, spray drying, fluid-bed drying, filter mat drying, or rotary drum drying. In some embodiments, the dried ERX-crystals can be milled to generate a desired particle size.

The composition of the resulting crystallized steviol glycosides comprises one or more of the steviol glycosides described herein, including stevioside, Reb B, Reb G, Reb C. Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The crystals may comprise, in an example, Reb D, Reb E, and Reb A. Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E. and Reb A may comprise between about 70% and about 100%, between about 72% and about 98%, between about 74% and about %%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

In embodiments, the mother liquor can be further processed at sub process 1710 of method 1700. Outputs from sub process 1710, which is described elsewhere herein, can be optionally provided as recycle stream inputs to the starting composition.

Each of the isolation at step 1706 and the processing of the mother liquor at sub process 1710 generate products. In particular, ERX-crystals are generated by step 1706. The types of ERX-crystals produced are based on the starting composition and the at least one enzyme used.

The ERX-crystals generated at step 1706 can be further processed in steps 1707-1709 of method 1700. In embodiments, the ERX reaction product can be processed to enhance the steviol glycoside purity prior to separation of the ERX reaction product crystals. As shown at step 1707 of method 1700, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C. and about 40° C., between about 40° C., and about 50° C., between about 50° C., and about 60° C., between about 60° C., and about 70° C., between about 70° C., and about 80° C., between about 80° C. and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1506 of method 1500 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

Crystal reformation can be performed via cooling crystallization and/or evaporation crystallization at step 1708 of method 1700. Mixing may be applied, as needed. In embodiments, the cooling crystallization comprises cooling via heat transfer surface cooling, evaporative cooling, or a combination thereof. In embodiments, the cooling includes significant evaporation and reducing the temperature to less than or equal to that of the ERX reaction temperature, thereby recovering the product crystals. The reformed crystals can then be isolated as purified ERX-crystals at step 1709 of method 1700 by separation and/or washing.

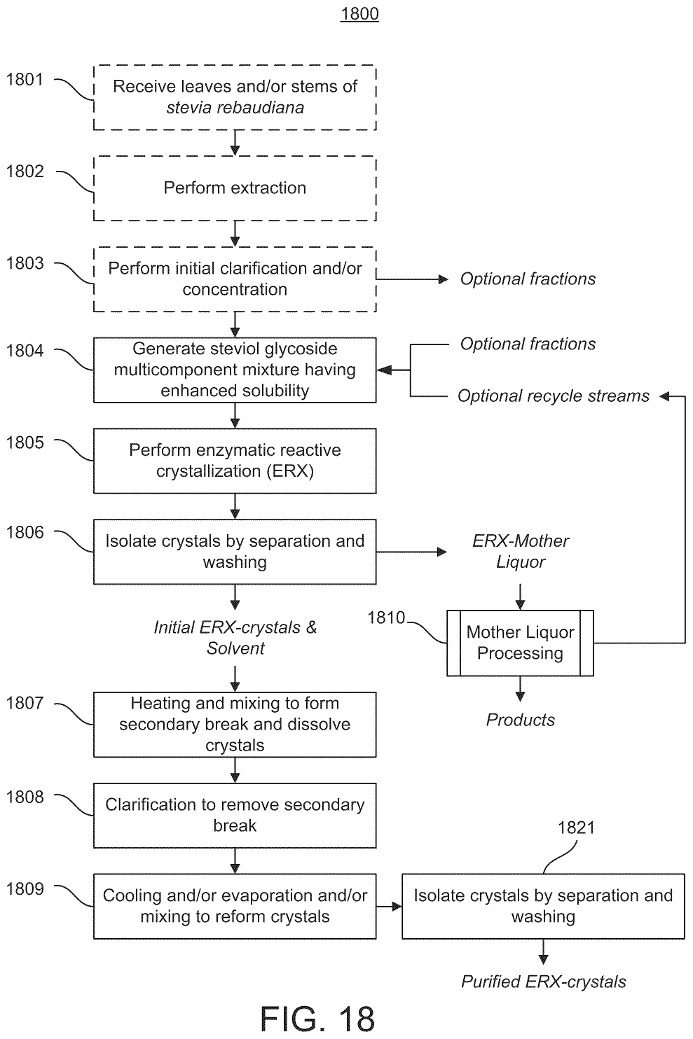

A variation of method 1700 will now be described with reference to method 1800 of FIG. 18.

At optional step 1801 of method 1800, leaves and/or stems of the Stevia rebaudiana may be received. At optional steps 1802 and 1803 of method 1800, the leaves and/or stems of the Stevia rebaudiana may be processed to extract steviol glycosides and to perform initial clarification and/or concentration thereof. Fractions may be obtained during this processing and may be reintroduced at step 1805, as shown in FIG. 18.

At step 1804 of method 1800, a starting composition, or steviol glycoside mixture having enhanced solubility, can be generated from the processed Stevia leaf extract. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 30) g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 1805 of method 1800, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides.

FIG. 13 is a flow diagram illustrating the role of each of B12GT and B13GT within a stevioside to Reb M pathway. In embodiments, the B12GT may be add a beta-linked glucose monomer to the C2′ of the 13-O-glucose and/or 19-O-glucose of a steviol glycoside substrate. For example, the B13GT may use an ADP-glucose sugar donor convert Stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may add a beta-linked glucose monomer to the C3′ of the 13-O-glucose and/or 19-O-glucose of a steviol glycoside substrate. For example, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

After ERX is performed at step 1805, the crystals are isolated at step 1806 of method 1800 by separation and/or washing. In embodiments, the result of ERX comprises crystallized steviol glycosides and a “mother liquor”, which comprises the buffer and any steviol glycosides that remain soluble. In embodiments, the precipitated steviol glycosides can be separated from the mother liquor by appropriate means including but not limited to vacuum filtration, centrifugation, and chamber filter press. In embodiments, the precipitated steviol glycosides can be further processed to enhance the purity of the precipitated steviol glycosides. The precipitated steviol glycosides can be washed with water to remove hydrophilic reaction components, such as residual sugars and salts. In embodiments, the precipitated steviol glycosides can be washed with an aqueous alcohol solvent to remove impurities. In embodiments, recovered and processed precipitated steviol glycosides can be further dried to form a powder. The precipitated steviol glycosides can be dried by any standard method including but not limited to freeze-drying, vacuum tray drying, spray drying, fluid-bed drying, filter mat drying, or rotary drum drying. In some embodiments, the dried ERX-crystals can be milled to generate a desired particle size.

The composition of the resulting crystallized steviol glycosides comprises one or more of the steviol glycosides described herein, including stevioside, Reb B, Reb G, Reb C. Reb F, Reb A, Reb I, Reb E, Reb E2, Reb AM, Reb H, Reb L, Reb K, Reb J, Reb M, Reb D. Reb N, Reb O, Reb Q, and synthetic steviol glycosides. The crystals may comprise, in an example, Reb D, Reb E, and Reb A. Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E, and Reb A may comprise between about 70% and about 100%, between about 72% and about 98%, between about 74% and about %%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

In embodiments, the mother liquor can be further processed at sub process 1810 of method 1700. Outputs from sub process 1810, which is described elsewhere herein, can be optionally provided as recycle stream inputs to the starting composition.

Each of the isolation at step 1806 and the processing of the mother liquor at sub process 1810 generate products. In particular, ERX-crystals are generated by step 1806. The types of ERX-crystals produced are based on the starting composition and the at least one enzyme used.

The ERX-crystals generated at step 1806 can be further processed in steps 1807-1809 and 1821 of method 1800. In embodiments, the ERX reaction product can be processed to enhance the steviol glycoside purity prior to separation of the ERX reaction product crystals. As shown at step 1807 of method 1800, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C., and about 40° C., between about 40° C., and about 50° C., between about 50° C. and about 60° C., between about 60° C., and about 70° C., between about 70° C., and about 80° C., between about 80° C., and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1807 of method 1800 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

At step 1808 of method 1800, a clarification can be performed to remove the hot break (i.e., remove impurities). In embodiments, clarifying comprises centrifugation, filtration, or a combination thereof. In embodiments, the hot break comprises proteins and other cellular derived debris. In embodiments, the clarification can comprise removing a solvent break. Crystal reformation can be performed via cooling crystallization and/or evaporation crystallization at step 1809 of method 1800. Mixing may be applied, as needed. In embodiments, the cooling crystallization comprises cooling via heat transfer surface cooling, evaporative cooling, or a combination thereof. In embodiments, the cooling includes significant evaporation and reducing the temperature to less than or equal to that of the ERX reaction temperature, thereby recovering the product crystals. The reformed crystals can then be isolated as purified ERX-crystals at step 1821 of method 1800 by separation and/or washing.

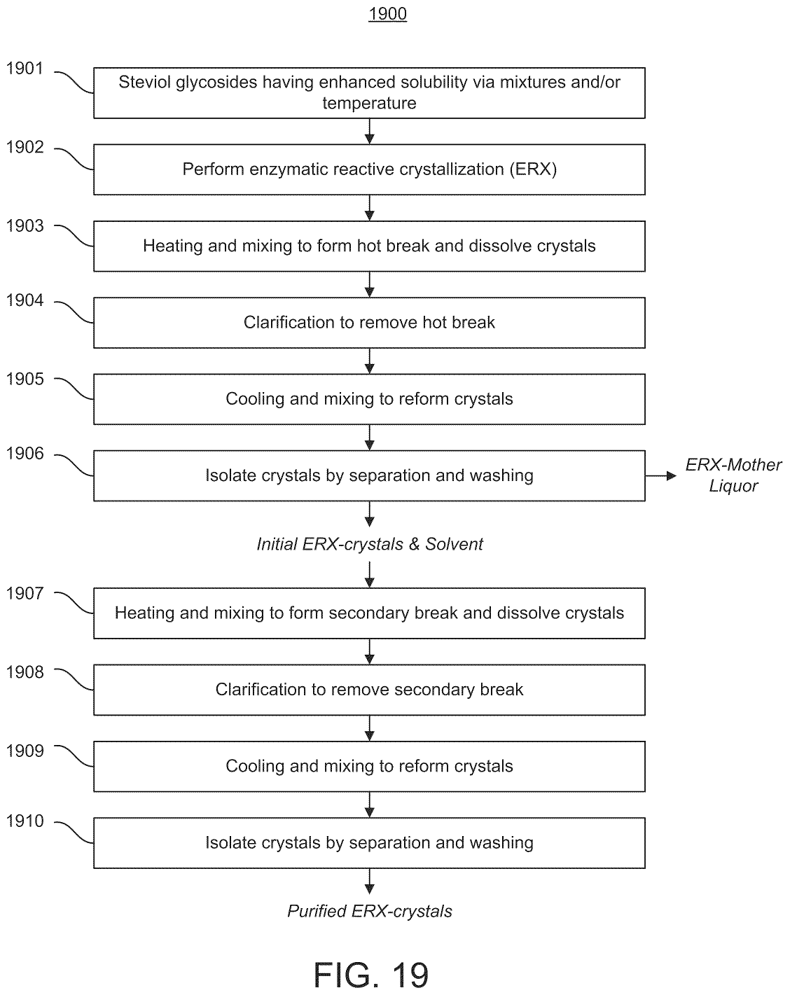

A variation of method 1800, wherein purification is performed before and after ERX, will now be described with reference to method 1900 of FIG. 19.

At step 1901 of method 1900, a starting composition, or steviol glycoside mixture having enhanced solubility, can be provided from the processed Stevia leaf extract. In some embodiments, a concentrated, crude Stevia leaf hot water extract can be provided as the starting composition. In embodiments, the steviol glycoside mixture may be a steviol glycoside single- or multi-component mixture. In embodiments, the starting composition comprises one or more steviol glycosides. In embodiments, the starting composition may comprise one of RA20, RA40, RA50, and/or RA60. In an embodiment, the starting composition may comprise at least 50 g/L RA50, at least 100 g/L RA50, at least 150 g/L RA50, at least 200 g/L RA50, at least 250 g/L RA50, at least 300 g/L RA50, at least 350 g/L RA50, at least 400 g/L RA50, at least 450 g/L RA50, at least 500 g/L RA50, at least 550 g/L RA50, at least 600 g/L RA50, at least 650 g/L RA50, at least 700 g/L RA50, at least 750 g/L RA50, at least 800 g/L RA50, at least 850 g/L RA50, at least 900 g/L RA50, at least 950 g/L RA50, or at least 1000 g/L RA50. Alternatively, the starting composition may be obtained as an already processed version of a Stevia rebaudiana. Moreover, the starting composition can be synthetic or at least partially purified, commercially available, or otherwise prepared.

At step 1902 of method 1900, the starting composition can be reacted with a reaction composition to perform enzymatic reactive crystallization (ERX). In embodiments, the reaction composition comprises at least one enzyme. In some embodiments, a crude enzyme mixture, a partially purified enzyme mixture, and/or a purified enzyme mixture can be used. In embodiments, the at least one enzyme described herein are prepared by expression in a host microorganism. Suitable host microorganisms include, but are not limited to, E. coli, Saccharomyces sp., Aspergillus sp., Pichia sp., and Bacillus sp. In embodiments, the at least one enzyme is expressed in E. coli. In embodiments, the at least one enzyme is expressed in Pichia pastoris. In embodiments, the at least one enzyme can be heat resistant. In embodiments, the at least one enzyme can have enhanced solubility.

In embodiments, the at least one enzyme comprises one or more glycosyltransferases. The one or more glycosyltransferases may be a wild type glycosyltransferase, a non-natural, engineered glycosyltransferase, a polypeptide having glycosyltransferase activity, or a combination thereof. In embodiments, the one or more glycosyltransferases may comprise a β-1,2-glycosyltransferase, a β-1-3-glycosyltransferase, and combinations thereof. The β-1,2-glycosyltransferase (B12GT) and/or the β-1,3-glycosyltransferase (B13GT) may use ADP-glucose as a sugar donor to convert steviol glycosides.

FIG. 13 is a flow diagram illustrating the role of each of B12GT and B13GT within a stevioside to Reb M pathway. In embodiments, the B12GT may be add a beta-linked glucose monomer to the C2′ of the 13-O-glucose and/or 19-O-glucose of a steviol glycoside substrate. For example, the B13GT may use an ADP-glucose sugar donor convert Stevioside to Reb E and Reb A to Reb D. In embodiments, the B12GT may be a non-natural, engineered B12GT glycosyltransferase as described in International Patent Application No. PCT/US2022/016820, which is incorporated herein by reference in its entirety. In embodiments, the B13GT may add a beta-linked glucose monomer to the C3′ of the 13-O-glucose and/or 19-O-glucose of a steviol glycoside substrate. For example, the B13GT may use an ADP-glucose sugar donor convert Reb E to Reb D and Red D to Reb M. In embodiments, the B13GT may be a non-natural, engineered B13GT glycosyltransferase as described in International Patent Application No. PCT/US2023/073344, which is incorporated herein by reference in its entirety.

The ERX-crystals generated at step 1902 can be further processed in steps 1903-1906 of method 1900. In embodiments, the ERX reaction product can be processed to enhance the steviol glycoside purity prior to separation of the ERX reaction product crystals. As shown at step 1903 of method 1900, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C. and about 40° C., between about 40° C., and about 50° C., between about 50° C., and about 60° C., between about 60° C., and about 70° C., between about 70° C., and about 80° C., between about 80° C. and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1903 of method 1900 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

At step 1904 of method 1900, a clarification can be performed to remove the hot break (i.e., remove impurities). In embodiments, clarifying comprises centrifugation, filtration, or a combination thereof. In embodiments, the hot break comprises proteins and other cellular derived debris. In embodiments, the clarification can comprise removing a solvent break. Crystal reformation can be performed via cooling crystallization and/or evaporation crystallization at step 1905 of method 1900. In embodiments, the cooling crystallization comprises cooling via heat transfer surface cooling, evaporative cooling, or a combination thereof. In embodiments, the cooling includes significant evaporation and reducing the temperature to less than or equal to that of the ERX reaction temperature, thereby recovering the product crystals. The reformed crystals can then be isolated as purified ERX-crystals at step 1906 of method 1900 by separation and/or washing.

After ERX is performed and initial purification is performed at steps 1902-1906, the ERX-crystals can be further processed in steps 1907-1910 of method 1900 to perform a second purification. In embodiments, the ERX reaction product can be processed to enhance the steviol glycoside purity prior to separation of the ERX reaction product crystals. As shown at step 1907 of method 1900, this may involve heating the reaction mixture to re-dissolve the ERX product, thereby providing formation of a hot break. In embodiments, the heating may comprise heating the reaction mixture to a temperature between about 10° C., and about 150° C., between about 10° C., and about 20° C., between about 20° C., and about 30° C., between about 30° C. and about 40° C., between about 40° C., and about 50° C., between about 50° C., and about 60° C., between about 60° C., and about 70° C., between about 70° C., and about 80° C., between about 80° C. and about 90° C., between about 90° C., and about 100° C., between about 100° C., and about 110° C., between about 110° C., and about 120° C., between about 120° C., and about 130° C., between about 130° C., and about 140° C., and between about 140° C., and about 150° C. In an embodiment, the mixture may be heated to a temperature greater than about 78° C. In embodiments, the mixture can be heated at step 1907 of method 1900 for a duration of between about 0.1 hours and about 10 hours, between about 0.1 hours and about 9.5 hours, between about 30 minutes and about 9 hours, between about 1 hour and about 8.5 hours, between about 2 hours and about 8 hours, between about 3 hours and about 7.5 hours, between about 4 hours and about 7 hours, and between about 5 hours and about 6.5 hours. In embodiments, the heating can be performed for a duration of about 6 hours. In embodiments, the heating temperature and heating duration is such that proteins are precipitated.

At step 1908 of method 1900, a clarification can be performed to remove the hot break (i.e., remove impurities). In embodiments, clarifying comprises centrifugation, filtration, or a combination thereof. In embodiments, the hot break comprises proteins and other cellular derived debris. In embodiments, the clarification can comprise removing a solvent break. Crystal reformation can be performed via cooling crystallization and/or evaporation crystallization at step 1909 of method 1900. Mixing may be applied, as needed. In embodiments, the cooling crystallization comprises cooling via heat transfer surface cooling, evaporative cooling, or a combination thereof. In embodiments, the cooling includes significant evaporation and reducing the temperature to less than or equal to that of the ERX reaction temperature, thereby recovering the product crystals. The reformed crystals can then be isolated as purified ERX-crystals at step 1921 of method 1900 by separation and/or washing.

The composition of the resulting crystallized steviol glycosides comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The crystals may comprise, in an example, Reb D, Reb E, and Reb A. Reb D, Reb E, and Reb A may each comprise between about 0% and about 100% of the crystal. Each of Reb D, Reb E, and Reb A may comprise between about 0.1% and about 20%, between about 0.2% and about 18%, between about 0.3% and about 16%, between about 0.4% and about 14%, between about 0.5% and about 12%, between about 0.6% and about 10%, between about 0.7% and about 8%, between about 0.8% and about 6%, between about 0.9% and about 4%, or between about 1% and about 2% of the crystal. Each of Reb D, Reb E, and Reb A may comprise between about 10% and about 90%, between about 15% and about 85%, between about 20% and about 80%, between about 25% and about 75%, between about 30% and about 70%, between about 35% and about 65%, between about 40% and about 60%, between about 45% and about 55%, between about 47.5% and about 52.5%, or between about 48% and about 52% of the crystal. Each of Reb D, Reb E, and Reb A may comprise between about 70% and about 100%, between about 72% and about 98%, between about 74% and about 96%, between about 76% and about 94%, between about 78% and about 92%, between about 80% and about 90%, between about 82% and about 88%, and between about 84% and about 86% of the crystal. In an example, the crystals comprise, on an anhydrous basis, 70.9% Reb D, 17.4% Reb E, and 0.4% Reb A. The resulting composition of steviol glycosides in the mother liquor comprises one or more of the steviol glycosides described herein, including stevioside, Reb B, Reb G, Reb C, Reb F, Reb A, Reb I, Reb E, Reb E2, Reb AM, Reb H, Reb L, Reb K, Reb J, Reb M, Reb D, Reb N, Reb O, Reb Q, and synthetic steviol glycosides. The mother liquor may comprise, in an example, Reb E, Reb D, Reb A, and stevioside at a ratio of 12.2:8.1:1:1.3. However, the ratio between the concentration of each steviol glycoside are highly variable and dependent upon the steviol glycoside(s) included within the reaction.

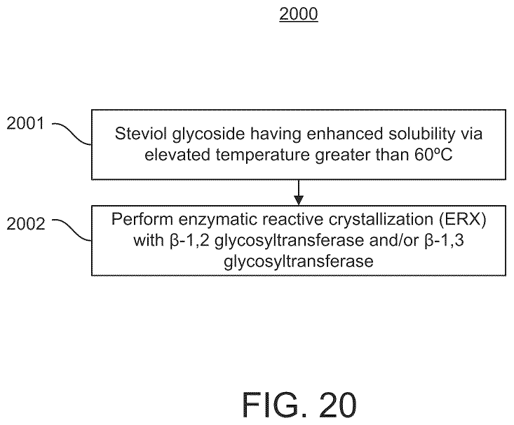

Referring now to FIG. 20, in the event the starting composition only comprises a single steviol glycoside, such as stevioside, other means for enhancing solubility can be deployed. As at step 2001 of method 2000, the starting composition can be heated to a temperature of greater than about 40° C., about 50° C., about 60° C., about 70° C., about 80° C., about 90°, and/or about 100° C. ERX can then be performed at step 2002 of method 2000 according to the methods described herein.

### EXAMPLES

### Example 1—ERX Reaction to Produce Rebaudioside D-Rich Product “ERX-D”

A 1 L spinner flask was used as a reactive crystallizer. A Pichia strain was grown in a 1-liter fermenter to express an engineered SuSy (SEQ ID NO: 1). A second Pichia strain was grown in another 1-liter fermenter to express an engineered B12GT (SEQ ID NO: 2). In each case, the cells were collected and mechanically lysed with a French press. The expressed protein from the lysate was partially purified by heat-treatment, centrifugation to remove coagulated proteins and cellular debris, and decolorized by treatment with a hydrophobic resin. The engineered SuSy and B12GT were reacted with a starting composition comprising 100 g/L RA50 (Stevia leaf extract with >95% steviol glycosides, >50% Reb A and >30% stevioside by mass), 100 g/L sucrose, and 0.5 mM ADP in 50 mM phosphate buffer (pH 6) and 250 mM NaCl. The reaction was conducted at 60° C. for 6 hours. The enzymatically mediated reactive crystallization yielded a precipitate and a mother liquor. The reaction was stored and chilled (2-8° C.) overnight. The precipitate was recovered by centrifugation, washed three times with water and dried in a vacuum oven. The dried reaction precipitate comprised 75.5% Reb D, 18.2% Reb E, 0.4% Reb A on an anhydrous basis. The ratio of major steviol glycosides in the mother liquor was measured to be 12.2:8.1:1:1.3 RebE:RebD:RebA:Stev.

### Example 2—Production and Characterization of ERX Crystals in 150-Liter Reactor

### Example 2a—Preparation of Reaction Enzymes

Two 150-liter scale fermentation runs were performed using two distinct engineered Pichia strains. One strain expressed an engineered SuSy enzyme (SEQ ID NO: 1). The other Pichia strain expressed an engineered B12GT enzyme (SEQ ID NO: 2). After the fermentations completed, the fermentation broths were combined, diluted, and centrifuged to isolate the cells from the fermentation broth to give 185 kg and diluted to reach 500 L total. The cells were then separated using a disc centrifuge to give 115.46 kg of cell cake.

The cell cake was resuspended 2:1 in buffer solution and the cells were lysed with 5 passes through a homogenizer. The resultant lysed cell stream was pasteurized in a flow system and then rapidly cooled. The liquid stream was further diluted and clarified using a disc centrifuge to remove the coagulated proteins and unwanted cell debris. The clear liquid was polished with a suitable dead-end filtration system and then stored overnight in a cooled tank. The liquid enzyme solution was then polished using a fixed bed resin treatment. This was followed by ultrafiltration to concentrate the enzyme mix and diafiltration to exchange the buffer solution from the lysis buffer to the reaction buffer.

### Example 2b—Enzymatically Mediated Reactive Crystallization

For the enzymatically mediated reactive crystallization process, a conical tank with a maximum working volume of 200 liters with a hot water jacket was used. Agitation was provided by a single overhead driven agitator shaft that had one small diameter screw impellor at the bottom in addition to two simple flat blade turbines, each with two blades oriented at a 30-degree angle. The clarified enzymes from Example 2a were reacted with a starting composition comprising 100 g/L RA50 (Stevia leaf extract with >95% steviol glycosides, >50% rebaudioside A and >30% stevioside by mass), 100 g/L sucrose, and 0.5 mM ADP in 50 mM potassium phosphate buffer (pH 6) and 250 mM sodium acetate. Notably, the starting composition of mixed steviol glycoside fed to the enzyme reactive crystallization process has a higher solubility than any of the individual steviol glycosides alone.

The reaction was carried out for 6 hours at 60° C. The system quickly reached the target reaction and primary crystallization temperature set point due to the mixing process. Gentle stirring was maintained for the time course of the reaction. After 6 hours of reaction, the system had reached 18% volume suspended solids of crystals. FIG. 8 shows the kinetics of crystal formation and FIG. 9 shows the kinetics of steviol glycoside conversion.

The system was then cooled with chilled water and stored for 20 hours to allow for additional crystal growth. The crystals were recovered using a chamber filter press and air blow dried. A total of 19 kg of blow-dried crystals were produced. These crystals were vacuum tray dried at 55° C. with the vacuum pump working at 50 mbar absolute pressure. Final weight of dry crystals was 9.06 kg. The dried crystals comprised of 70.9% Reb D, 17.4% Reb E and 0.4% Reb A on an anhydrous basis.

### Example 2c—Mother Liquor Processing from Enzymatically Mediated Reactive Crystallization

The crystals from the enzymatically mediated reactive crystallization of Example 2b were recovered using a filter press and this gave a resultant clarified mother liquor. This mother liquor can be unique in that it can comprise steviol glycoside compositions that are especially soluble.

The mother liquor was clarified to remove enzymes by ultrafiltration and diafiltration to collect residual steviol glycosides present. This yielded 204 liters of combined ultrafiltration filtrate containing ratio of Reb E to Reb D of 3.56:1.

The soluble steviol glycoside solution was loaded onto a non-ionic resin by adsorption. The column was then washed with water to remove the hydrophilic reaction components. The rebaudiosides were then eluted using 70% isopropyl alcohol in water at room temperature. The first 1.5 bed volumes contained 77.14% of the steviol glycosides by mass, the next bed volume contained about 1% and the last 1.5 bed volumes contained 0.03%.

The 348 liters of eluant was evaporated sequentially down to 20 liters. At this point water was added as a solvent chase to help strip the last alcohol. The eluent mix was then concentrated to 57% dissolved solids and the concentrate was transferred to trays in preparation for vacuum tray drying. Immediate crystallization occurred at the surface when cooling down, illustrating that evaporative and/or cooling crystallization can be used to generate crystals from the mother liquor, yet may require relatively high concentrations. The steviol glycoside concentrate was vacuum tray dried to obtain 4 kg of final dry crystals. The crystals were primarily composed of Reb D and Reb E in a ratio of 1:3.4. The crystals were readily soluble in water.

### Example 3—Low Solubility of Mixed Rebaudioside A and Rebaudioside D

Compositions representative in part of a time point in enzymatically mediated reactive crystallization were prepared as follows. 100 gram of deionized water was added to 4 grams of sucrose and 2.17 grams of fructose and the mixture heated to 60° C. with agitation. Rebaudioside D (0.50 gram) and rebaudioside A (0.513 gram) were added. The final mixture composition is 0.48% rebaudioside A and 0.45% rebaudioside D by weight. White solids remain present and the mixed rebaudiosides are not soluble even at a total of 0.93% by weight.

### Example 4—High Solubility of Mixed Rebaudioside A and Stevioside

A rebaudioside mixture representative of a partially purified leaf extraction was prepared as follows: 200 grams of RA50 (Stevia leaf extract with >95% steviol glycosides, >50% rebaudioside A and >30% stevioside by mass) was dissolved in 800 ml water with heating and mixing at 80° C. At 60° C., this mixture was clear demonstrating the high solubility of a mixture of Reb A and stevioside.

### Example 5—Concentration of ERX Mother Liquor to Yield Additional Crystals

Example 5 illustrates the principle of treatment of ERX mother liquor by concentration to yield additional crystals. The reaction mother liquor rich in Reb D and Reb E from Example 1 was purified to remove enzymes, sugar and ADP via ultrafiltration, adsorption, desorption, and desolventization. The resultant material contained essentially all the steviol glycosides that were present in the ERX mother liquor. This material was dried to allow experimentation with exact levels of dissolved solids (DS). This material was dissolved in water at a rate of 1 gram of dissolved material per 10 gram of water. Complete solubility was observed, which is consistent with ERX mother liquor for mixed rebaudiosides being of high solubility. Analysis indicated that the ERX mother liquor rich in both rebaudioside E and rebaudioside D. Upon further evaporation of this water-soluble mixture and cooling, fresh crystals formed.

### Example 6—ERX Preparation of Rebaudioside M, Hot Break Clarification, Crystal Reformation, Redissolving & Recrystallization

Example 6 illustrates preparation of enzymes, formation of Reb M by ERX, formation of hot break, removal of hot break, and crystal reformation by cooling crystallization.

### Example 6a—Preparation of Reaction Enzymes

Three 1-liter scale fermentation runs were performed using three distinct engineered Pichia strains. One strain expressed an engineered SuSy enzyme (SEQ ID NO: 1).

Another Pichia strain expressed an engineered B12GT enzyme (SEQ ID NO: 2). Another Pichia strain expressed an engineered B13GT enzyme (SEQ ID NO: 3). After the fermentations completed, the fermentation broths were individually centrifuged to isolate the cells from the fermentation broth. Each fermentation produced significant amounts of wet cell cake.

Cell cake from above was combined at ratio of 1:1:2 of wet cell weight of the SuSy Pichia:B12GT Pichia:B13GT Pichia. This mixture was resuspended 2:1 in a sodium acetate buffer solution and the cells were lysed with 5 passes through a high-pressure homogenizer. The resultant lysed cell stream was pasteurized and rapidly cooled in a continuous flow pasteurizer. The liquid stream was further diluted and clarified using a centrifuge to remove the coagulated proteins and unwanted cell debris. The clear liquid was stored overnight at 4′C. The clear liquid was concentrated and buffer exchanged by Ultrafiltration. A total of 1370 ml of concentrate was generated. A sample of this was diluted 570/730 fold, and 400 gram of this enzyme mixture was taken and used here.

### Example 6b—ERX Reaction

The 400 gram of enzyme mixture from Example 6a was combined with 400 ml of a steviol glycoside-based composition. The resultant 800-gram mixture contained 10 g/L RA50 (Stevia leaf extract with >95% steviol glycosides, >50% rebaudioside A and >30% stevioside by mass), 50 g/L sucrose, and 1 mM ADP in 50 mM phosphate buffer (pH 6) with 250 mM sodium acetate. The reaction was preheated for more than 1 hour and reacted at 56.7° C. for 5 hours and gave a precipitate and a mother liquor.

### Example 6c—Preparation of Hot Break

The reaction mixture above was heated in the reaction flask for 30 minutes at 86° C. Then it was cooled to 75° C., and filtered to remove the hot break. The clarified filtrate was 802.61 grams. The hot break has a mass of 1.73 grams. The filtered ERX product was chilled for 12 hours to 4° C. to allow the ERX crystals to reform. The ERX crystals were recovered on a filter and washed with three aliquots of ice cold water with a total wash mass used of 68.3 grams. The mass of initial ERX product crystals after drying that was recovered was 3.67 grams. The rate of crystal growth was modest at 4′C and addition time allowed an additional 3.96 grams of dry crystals to be recovered after washing. The combined crystals had the following analysis—

| Type of Rebaudioside | Percent of Composition |
|---|---|
| AM | 0.5% |
| B | 0.7% |
| B isomer | 0.5% |
| D | 0.3% |
| I | 0.4% |
| M | 96.9% |
| Other | 0.7% |

### Example 6d—Recrystallization

The combined crystals above were dissolved in 323 grams of pure water at 70′C and then allowed to cool and recrystallize overnight. A mass of 2.43 grams of dry crystals was recovered after washing. The crystals had the following analysis—

| Type of Rebaudioside | Percent of Composition |
|---|---|
| AM | 0.5% |
| B | 0.7% |
| B isomer | 0.3% |
| D | 0.1% |
| I | 0.2% |
| M | 97.8% |
| Other | 0.3% |

### Example 7—Mother Liquor Processing and Steviol Glycoside Recycling

Example 7 illustrates mother liquor processing and recycling of steviol glycosides within an ERX reaction system.

### Example 7a—Preparation of Reaction Enzymes

SEQ ID NO: 1, SEQ ID NO: 2, and SEQ ID NO: 3 from Example 6a, at an enzyme dosing of 6.1 mg enzyme per gram lyophilized material, were combined with 60 g/L RA60 (Stevia leaf extract with >95% steviol glycosides, >60% rebaudioside A and >20% stevioside by mass), 250 g/L sucrose, and 1 mM ADP at 56° C. Three successive reactions were carried out. First, 100 ml of the composition was placed into a rotary evaporator. An immersion blender was used at 2 hours, 2.5 hours, and 3 hours to break up custard. Crystals comprised about 25% to 30% of the volume. The estimated resulting mother liquor volume was 70 ml. Next, 7.8 of 600 g/L RA60 was combined with 70 ml of the mother liquor in the rotary evaporator. The immersion blender was used as above. The estimated resulting mother liquor volume was 56 ml. Finally, 5.6 ml of 600 g/L RA60 was combined with 50 ml of the processed mother liquor in the rotary evaporator. The immersion blender was used as above. Total protein concentration was tracked throughout the experiment. At timer intervals, samples were collected for HPLC analysis of crystal formation and composition.

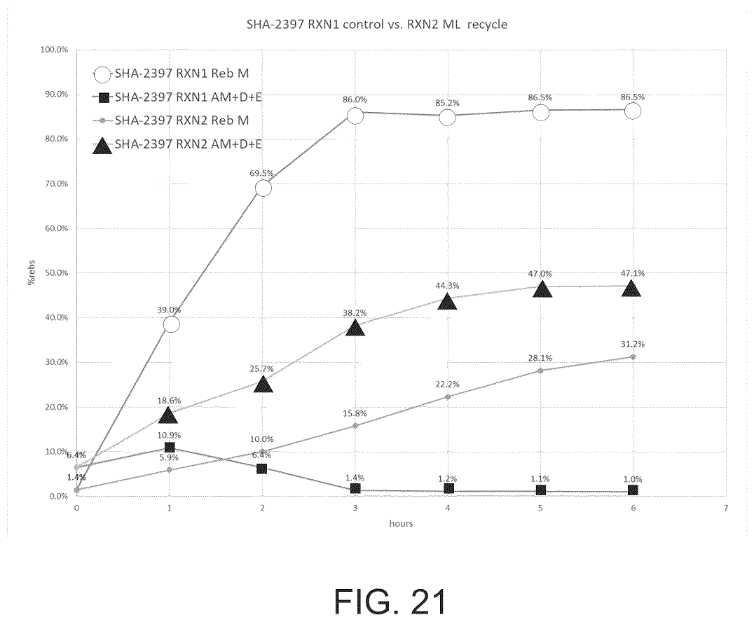

This data is shown in FIG. 21, which is a graphical representation of mother liquor recycling. FIG. 21 illustrates high-performance liquid chromatography results showing a RXN1 control having 86% Reb M at hour 3, which can be compared with a RXN2 sample, with recycled mother liquor, having a slower rate of Reb M formation. Regarding a RXN2 sample, with recycled mother liquor, Reb M reached 31% at hour 6, and Reb AM, Reb D, and Reb E reached 47% at hour 6. Regarding the RXN1 control, Reb AM, Reb D, and Reb E reached 1% by hour 3.

### Example 7b—Steviol Glycoside Generation

SEQ ID NO: 1. SEQ ID NO: 2, and SEQ ID NO: 3 from Example 6a, at an enzyme dosing of 6.1 mg enzyme per gram lyophilized material, were combined with 60 g/L RA60 (Stevia leaf extract with >95% steviol glycosides, >60% rebaudioside A and >20% stevioside by mass), 250 g/L sucrose, and 1 mM ADP at 56° C. Three successive reactions were carried out. First, a 150 ml volume of the composition was placed into a first rotary evaporator and an additional 50 mL volume of the composition was placed into a second rotary evaporator as a control. The first rotary evaporator contained the enzymes and reactants. An immersion blender was used at 2 hours, 2.5 hours, and 3 hours to break up custard. Crystals comprised about 25% to 30% of the volume. The estimated resulting mother liquor volume was 70 ml. The second rotary evaporator contained reconstituted enzyme without reactants added or immersion blender. The reconstituted enzyme was still subjected to heating and the gentle shear of the rotary evaporator. Next, 75 mL of the mother liquor was run through a sephadex column to remove excess fructose (confirm removal using fructose assay). Protein concentration was tested. Each solution (as well as the control solution) was then diluted with additional buffer to bring up to the same concentration. 5×9 mL aliquots were then prepared from the reserved, untreated mother liquor. Each aliquot was supplemented with additional reagent according to the following: (1) Prepare another 9 mL aliquot from the solution treated with the sephadex filter; (2) To the solution from the above reaction, add 12.5 g sucrose and 2 mM ADP; and (3) Prepare a 9 mL aliquot of the solution. All prepared 9 mL aliquots were reacted using a 10 mL bioconversion QC setup. The reaction was initiated by adding 1 mL of 600 g/L RA60. Samples for HPLC were obtained every hour for four hours and tested for rebaudiosides.

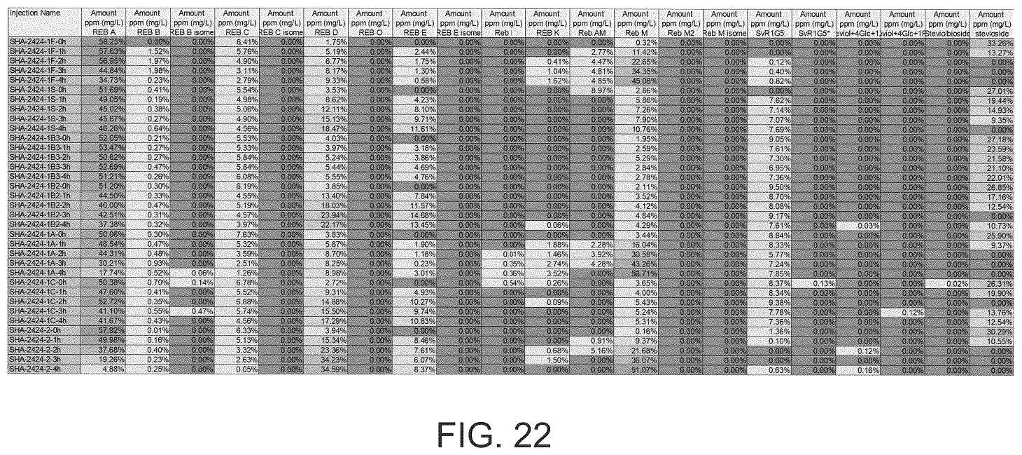

FIG. 22 shows the amount of rebaudiosides generated at each timepoint for a myriad conditions identified under the “Injection Name” column. Samples marked 1A correspond to supplementary reagent ADP, samples marked 1B2 correspond to supplementary reagent β12GT, samples marked 1B3 correspond to supplementary reagent β13GT, and samples marked 1S correspond to supplementary reagent sucrose. Moreover, 1C refers to a control which underwent a first reaction under standard conditions and then was used for a second reaction without any treatment. 1A refers to an ADP test which underwent a first reaction under standard conditions and then was supplemented with additional ADP prior to being used for a second reaction. 1F refers to a fructose test, which underwent a first reaction under standard conditions and then was treated to remove excess fructose prior to being used for a second reaction. 1B2 refers to an β12GT test which underwent a first reaction under standard conditions and then was supplemented with additional β12GT prior to being used for a second reaction. 1BB refers to an β13GT test which underwent a first reaction under standard conditions and then was supplemented with additional β13GT prior to being used for a second reaction. 1S refers to a sucrose test which underwent a first reaction under standard conditions and then was supplemented with additional sucrose prior to being used for a second reaction. 2 refers to an absolute control, which did not undergo a first reaction and was not subjected to high shear, though it was subjected to the same temperature conditions as 1C, 1A, 1F, 1B2, 1B3, and 1S.

### Example 7c—Steviol Glycoside Generation by Retentate-Based Enzymes

β12GT, β13GT, and SuSy remaining within the above mother liquor was used as recycled enzymes for supplemental 10 ml bioconversions.

Specifically, reactions used two steviol glycoside feedstocks: RA6 powder and concentrated RA60 syrup at about 60 g/L. Additionally, two levels of Na-ADP dosage were used, one having an additional 1 mM Na-ADP. Reactions were carried out overnight without heat. After reacting, vials were warmed to 60° C., and sampled every 2 hours thereafter. Vials were finally sampled after 3 time points.

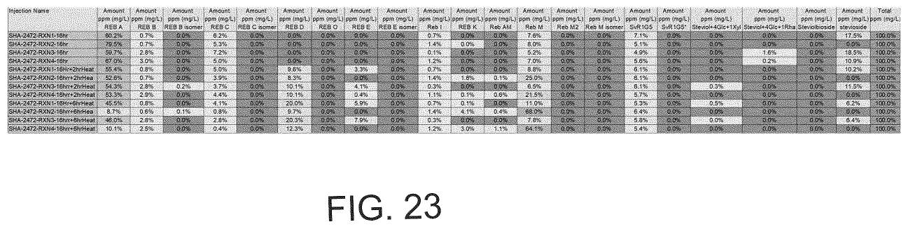

Referring to FIG. 23, steviol glycoside production from these reactions is shown. RXN1 was reacted with 60 g/L RA60 powder but without ADP. RXN2 was reacted with 60 g/L RA60 powder and with ADP. RXN3 was reacted with about 75 g/L RA60 syrup but without ADP. RXN4 was reacted with about 75 g/L RA60 syrup and with ADP.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

### NUMBERED EMBODIMENTS OF THE INVENTION

Notwithstanding the appended claims, the disclosure sets forth the following numbered embodiments:

(1) A method for generating and isolating steviol glycoside crystals, comprising performing enzymatically mediated reactive crystallization by reacting a starting composition comprising at least one soluble steviol glycoside with an enzyme composition comprising at least one enzyme configured to add one or more sugar monomers to the at least one soluble steviol glycoside, and isolating at least one steviol glycoside crystal produced by the reaction, wherein the isolated steviol glycoside crystal comprises at least one additional sugar monomer as compared to the at least one soluble steviol glycoside.

(2) The method of (1), w % herein the reacting comprises converting the at least one soluble steviol glycoside to a different steviol glycoside having a reduced solubility relative to the at least one steviol glycoside, thereby causing crystallization of the different steviol glycoside.

(3) The method of either (1) or (2), wherein the one or more sugar monomers comprises galactose, glucose, xylose, glucosamine, galactosamine, glucuronic acid, galactofuranose, mannose, fucose, rhamnose, acetylneuraminic acid, and mannooctanoic acid.

(4) The method of any one of (1) to (3), wherein the starting composition comprises two or more soluble steviol glycosides.

(5) The method of (4), wherein the two or more soluble steviol glycosides are present in the starting composition at a concentration of at least 100 gram per liter.

(6) The method of either (4) or (5), wherein the two or more soluble steviol glycosides comprise naturally-occurring steviol glycosides selected from the group consisting of: steviol-13-O-glucoside; steviol-19-O-glucoside; rubusoside; steviol-1,2-bioside; steviol-1,3-bioside; rubusoside, dulcoside B; dulcoside A; rebaudioside B; rebaudioside G; stevioside; rebaudioside C; rebaudioside F; rebaudioside A; rebaudioside I; rebaudioside E; rebaudioside E2; rebaudioside AM; rebaudioside H; rebaudioside L; rebaudioside K; rebaudioside J; rebaudioside M; rebaudioside X; rebaudioside D; rebaudioside N; rebaudioside O; rebaudioside Q; and enzymatically glycosylated steviol glycosides.

(7) The method of any one of (4) to (6), wherein the two or more soluble steviol glycosides have enhanced solubility.

(8) The method of any one of (1) to (7), wherein the starting composition comprises a Stevia leaf extract.

(9) The method of any one of (1) to (8), where the starting composition comprises stevioside and rebaudioside A.

(10) The method of any one of (1) to (9), wherein the starting composition comprises at least 30 g/L stevioside and at least 50 g/L rebaudioside A.

(11) The method of any one of (1) to (10), wherein the isolated steviol glycoside crystal comprises one or more of rebaudioside A, rebaudioside I, rebaudioside E, rebaudioside E2, rebaudioside AM, rebaudioside H, rebaudioside L, rebaudioside K, rebaudioside J, rebaudioside M, rebaudioside X, rebaudioside D, and enzymatically glycosylated steviol glycosides.

(12) The method of any one of (1) to (11), wherein the at least one enzyme configured to add the one or more sugar monomers to the at least one soluble steviol glycoside comprises one or more of a β-1,2-glycosyltransferase (B12GT) and a β-1,3-glycosyltransferase (B13GT).

(13) The method of (12), wherein the B12GT and/or the B13GT is an engineered enzyme.

(14) The method of any one of (1) to (13), wherein the at least one enzyme configured to add the one or more sugar monomers to the at least one soluble steviol glycoside utilizes a nucleotide-sugar (NDP-sugar) as a substrate.

(15) The method of (14), wherein the NDP comprises one of adenosine diphosphate (ADP), cytidine diphosphate (CDP), thymidine diphosphate (TDP), and guanosine diphosphate (GDP).

(16) The method of any one of (1) to (15), wherein the enzyme composition further comprises a sucrose synthase.

(17) The method of (16), wherein the sucrose synthase is capable of generating NDP-glucose.

(18) The method of (17), wherein the sucrose synthase is capable of generating ADP-glucose.

(19) The method of any one of (1) to (18), further comprising heating a reaction composition comprising the starting composition and the enzyme composition.

(20) The method of (19), wherein the reaction composition is heated to a predetermined reaction temperature greater than or equal to about 10° C., about 20° C., about 30° C., about 40° C., about 50° C., about 60° C., about 70° C., and about 80° C.

(21) The method of either (19) or (20), wherein the reaction composition comprises a pH of between about 4 and about 10, between about 5 and about 9, and between about 6 and about 8.

(22) The method of any one of (19) to (21), wherein the reaction composition further comprises a NDP.

(23) The method of (22), wherein the NDP comprises ADP. CDP, TDP, and/or GDP.

(24) The method of any one of (19) to (22), wherein the reaction composition further comprises a sugar.

(25) The method of (24), wherein the sugar comprises one or more of galactose, glucose, xylose, glucosamine, galactosamine, glucuronic acid, galactofuranose, mannose, fucose, rhamnose, acetylneuraminic acid, and mannooctanoic acid.

(26) The method of any one of (1) to (25), wherein the reacting the starting composition and the enzyme composition is performed for a predetermined duration of time.

(27) The method of (26), wherein the predetermined duration of time is at least about 6 minutes, about 30 minutes, about 1 hour, about 1.5 hours, about 2 hours, about 2.5 hours, about 3 hours, about 3.5 hours, about 4 hours, about 4.5 hours, about 5 hours, about 5.5 hours, and about 6 hours.

(28) The method of any one of (1) to (27), further comprising adding a secondary enzyme composition to the reaction composition during the enzymatically mediated reactive crystallization.

(29) The method of any one of (1) to (28), further comprising performing evaporation crystallization and/or cooling crystallization.

(30) The method of any one of (1) to (29), wherein isolating the at least one steviol glycoside crystal comprises recovering a mother liquor from the reaction, wherein the mother liquor comprises leftover soluble steviol glycosides.

(31) The method of (30), further comprising processing the mother liquor by clarifying and concentrating the leftover soluble steviol glycosides, and performing a second enzymatically mediated reactive crystallization on the leftover soluble steviol glycosides to produce second steviol glycoside crystals.

(32) The method of (31), further comprising performing evaporative crystallization and/or cooling crystallization.

(33) The method of (30) or (31), further comprising recycling the mother liquor into a subsequent starting composition.

(34) The method of any one of (1) to (33), further comprising, after the reacting, re-heating the reaction composition to form a hot break comprising impurities.

(35) The method of (34), further comprising clarifying the reaction composition by removing the hot break.

(36) The method of (35), further comprising performing evaporation crystallization and/or cooling crystallization on the clarified reaction composition.

(37) The method of any one of (1) to (36), further comprising heating the starting composition to a predetermined temperature.

(38) The method of (37), wherein the predetermined temperature is at least about 40°, about 50° C., about 60° C., about 70° C., about 80° C., about 90° C., and about 100° C.

(39) The method of any one of (1) to (38), wherein the isolating further comprises separating and washing a reaction composition comprising the starting composition and the enzyme composition.

(40) A crystal composition, comprising a steviol glycoside co-crystal having a ratio of rebaudioside E to rebaudioside D of between 1:3 and 1:5 on an anhydrous basis.

(41) A crystal composition, comprising a steviol glycoside co-crystal having at least 65% rebaudioside D and at least 10% rebaudioside E on an anhydrous basis.

(42) A crystal composition, comprising a steviol glycoside co-crystal having at least 10% rebaudioside D and at least 70% rebaudioside M on an anhydrous basis.

(43) A crystal composition, comprising rebaudioside E to rebaudioside D at a ratio between 2:1 and 5:1 on an anhydrous basis.

(44) A crystal composition comprising crystalline steviol glycoside species.

(45) A crystal composition, comprising at least 65% rebaudioside D and at least 10% rebaudioside E on an anhydrous basis.

(46) A crystal composition, comprising at least about 90% Reb M, about 91% Red M, about 92% Reb M, about 93% Reb M, about 94% Reb M, about 95% Reb M, about 96% Reb M, about 97% Reb M, about 98% Reb M, or about 99% Reb M on an anhydrous basis.

(47) The crystal composition of any one of claims 40-46 produced according to the method of any one of (1) to (46).

(48) A method, comprising spray-fragmenting the crystal composition of (47).

(49) A spray-fragmented composition comprising the crystal composition of any one of (40) to (46).

(50) A steviol glycoside composition comprising a mother liquor, the mother liquor having high solubility of steviol glycosides.

(51) The steviol glycoside composition of claim 48 produced according to the method of any one of (1) to (50).

(52) A beverage composition prepared from a starting composition comprising between 10 ppm and 1000 ppm of any of the crystal compositions of any one of (40) to (46).

(53) A baked food product prepared from a starting composition comprising between 10 ppm and 1000 ppm of any of the crystal compositions of any one of (40) to (46).

(54) The method of claim of either (30) or (50), wherein steviol glycosides within the mother liquor comprise, on average, at least at least 0.5, at least 1, at least 2, and/or at least 3 units of glucose more per steviol glycoside than the at least one soluble steviol glycoside of the starting composition.

(55) The method of any one of (1) to (54), wherein the at least one steviol glycoside crystal produced by the reaction comprises, on average, at least 0.5, at least 1, at least 2, and/or at least 3 units of glucose more per steviol glycoside than the at least one soluble steviol glycoside of the starting composition.

(56) The method of either (30) or (50), wherein the at least one soluble steviol glycoside comprises an average molecular weight lower than an average molecular weight of steviol glycosides within the mother liquor.

(57) The method of any one of (1) to (56), wherein the at least one enzyme comprises an engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 3, and wherein the polypeptide comprises H at position 24, D at position 123, D at position 379, and Q at position 380.

(58) The method of any one of (1) to (57), wherein the at least one enzyme comprises an engineered beta-1,2-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 2.

(59) The method of any one of (1) to (58), wherein the at least one enzyme comprises an engineered sucrose synthase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO; 1.

(60) A method for generating and isolating steviol glycoside crystals, comprising performing enzymatically mediated reactive crystallization by reacting a starting composition comprising at least one soluble steviol glycoside with an enzyme composition comprising at least one enzyme configured to add one or more sugar monomers to the at least one soluble steviol glycoside, isolating at least one steviol glycoside crystal produced by the reaction, wherein the isolated steviol glycoside crystal comprises at least one additional sugar monomer as compared to the at least one soluble steviol glycoside, and recovering a mother liquor produced by the reaction.

1. A method for generating and isolating steviol glycoside crystals, comprising:
performing enzymatically mediated reactive crystallization by reacting a starting composition comprising at least one soluble steviol glycoside with an enzyme composition comprising at least one enzyme configured to add one or more sugar monomers to the at least one soluble steviol glycoside; and
isolating at least one steviol glycoside crystal produced by the reaction, wherein the isolated steviol glycoside crystal comprises at least one additional sugar monomer as compared to the at least one soluble steviol glycoside.

2. The method of claim 1, wherein the reacting comprises converting the at least one soluble steviol glycoside to a different steviol glycoside having a reduced solubility relative to the at least one steviol glycoside, thereby causing crystallization of the different steviol glycoside.

3. The method of claim 1, wherein the one or more sugar monomers comprises galactose, glucose, xylose, glucosamine, galactosamine, glucuronic acid, galactofuranose, mannose, fucose, rhamnose, acetylneuraminic acid, and mannooctanoic acid.

4. The method of claim 1, wherein the starting composition comprises two or more soluble steviol glycosides.

5. The method of claim 4, wherein the two or more soluble steviol glycosides are present in the starting composition at a concentration of at least 100 gram per liter.

6. The method of claim 4, wherein the two or more soluble steviol glycosides comprise naturally-occurring steviol glycosides selected from the group consisting of: steviol-13-O-glucoside; steviol-19-O-glucoside; rubusoside; steviol-1,2-bioside; steviol-1,3-bioside; rubusoside; dulcoside B; dulcoside A; rebaudioside B; rebaudioside G; stevioside; rebaudioside C; rebaudioside F; rebaudioside A; rebaudioside I; rebaudioside E; rebaudioside E2; rebaudioside AM; rebaudioside H; rebaudioside L; rebaudioside K; rebaudioside J; rebaudioside M; rebaudioside X; rebaudioside D; rebaudioside N; rebaudioside O; rebaudioside Q; and enzymatically glycosylated steviol glycosides.

7. The method of claim 4, wherein the two or more soluble steviol glycosides have enhanced solubility.

8. The method of claim 1, wherein the starting composition comprises a Stevia leaf extract.

9. The method of claim 1, where the starting composition comprises stevioside and rebaudioside A.

10. The method of claim 1, wherein the starting composition comprises at least 30 g/L stevioside and at least 50 g/L rebaudioside A.

11. The method of claim 1, wherein the isolated steviol glycoside crystal comprises one or more of rebaudioside A, rebaudioside I, rebaudioside E, rebaudioside E2, rebaudioside AM, rebaudioside H, rebaudioside L, rebaudioside K, rebaudioside J, rebaudioside M, rebaudioside X, rebaudioside D, and enzymatically glycosylated steviol glycosides.

12. The method of claim 1, wherein the at least one enzyme configured to add the one or more sugar monomers to the at least one soluble steviol glycoside comprises one or more of a β-1,2-glycosyltransferase (B12GT) and a β-1,3-glycosyltransferase (B13GT).

13. (canceled)

14. The method of claim 1, wherein the at least one enzyme configured to add the one or more sugar monomers to the at least one soluble steviol glycoside utilizes a nucleotide-sugar (NDP-sugar) as a substrate.

15. (canceled)

16. The method of claim 1, wherein the enzyme composition further comprises a sucrose synthase.

17-18. (canceled)

19. The method of claim 1, further comprising heating a reaction composition comprising the starting composition and the enzyme composition.

20-28. (canceled)

29. The method of claim 1, further comprising performing evaporation crystallization and/or cooling crystallization.

30. The method of claim 1, wherein isolating the at least one steviol glycoside crystal comprises recovering a mother liquor from the reaction, wherein the mother liquor comprises leftover soluble steviol glycosides.

31. The method of claim 30, further comprising processing the mother liquor by clarifying and concentrating the leftover soluble steviol glycosides, and performing a second enzymatically mediated reactive crystallization on the leftover soluble steviol glycosides to produce second steviol glycoside crystals.

32-45. (canceled)

46. A crystal composition, comprising at least about 90% Reb M, about 91% Red M, about 92% Reb M, about 93% Reb M, about 94% Reb M, about 95% Reb M, about 96% Reb M, about 97% Reb M, about 98% Reb M, or about 99% Reb M on an anhydrous basis.

47-59. (canceled)

60. A method for generating and isolating steviol glycoside crystals, comprising:
performing enzymatically mediated reactive crystallization by reacting a starting composition comprising at least one soluble steviol glycoside with an enzyme composition comprising at least one enzyme configured to add one or more sugar monomers to the at least one soluble steviol glycoside;
isolating at least one steviol glycoside crystal produced by the reaction, wherein the isolated steviol glycoside crystal comprises at least one additional sugar monomer as compared to the at least one soluble steviol glycoside; and
recovering a mother liquor produced by the reaction.


SEQUENCE LISTING (3 sequences)

SEQ ID NO: 1 - protein, 793 aa, synthetic construct
MIEELRQQLLDHPNEWRAFLRHLVASGRPMWLHHDLWRACKDFREQPPEGYPEDIGWLEQFVRHTQEAIFRDPWMVFAWRLRPGVWEYVRIHVEQLAVEELSTDEYLQFKEQLVDGGAEGEAVLTVDFEDFRPGFQRLKDESTIGNGLEHLNRHLAGRMWTDLGQGRSQLLEFLRLHRLDGQQLMLSNRNTDFDSLRQAVQYLGTLPPDTPWAEFREDMRRRGFEPGWGDTAERVRETMRLLMDLLDSPSPAALEKFLARIPMISNVLIVSIHGWFAQDNVLGRPDTGGQVVYILDQARALEREMRERLRQQGVDVEPRILIATRLIPESDGTTCDQRLEPVSGTENVQILRVPFRYEDGNIHPHWISRFKVWPYLERYAEDLEREVLAELGGRPDLIIGNYSDGNLVATLLSEKLGVTQCNIAHALEKSKYLYSDLYWKDFEQQYHFACQFTADLIAMNAADIIITSTYQEIAGNDREVGQYESHQHYTLPGLYRVVNGIDVFDPKFNIVSPGADPRIYFSYSRTEKRFSSLHPEIEELLFGREPGPPHRGVLEDRDKPIILSMARLDRIKNLTGLAEWYGRSSRLRELANLVIIGGHVDVQNSTDREEREEMRRMHEIMDEYQLDGQMRWLGSQLDKTRVGELYRYVADTRGVFVQPALFEAFGLTVIEAMASGLPVFATRHGGPLEIIEDGVSGFHIDPNDPEAVAEKLADFLEAARERPKYWEKISDAALQRVRERYTWERYAERLMTLARVYGFWRFVSSRERQVTRRYLQMFRHLQWRPLAHAVPME

SEQ ID NO: 2 - protein, 440 aa, synthetic construct
QTQQVLMFPWLALGHVNPFLRIAKQLADRGFVIYLVSTAINLELAKKRIPYKYSNSIHLVEYALPELPELPPHLHTTNGLPPHLNGTLHKALKMSAPEFSKQVQNLKPDLVLYDFLIPWAEKVALSQGVPGVPLLTSGAALFSYFFNFLKRPGHEFPFPAIRLSKREQDKMREMFGTEPTELDFLVPANAGIMLMCTSRVIEAKYLDYCQKLTNVKVVPVGAPFQDPLTEDIYDPELIRWLDTKPPNSVVYVSFGSEYFLSREQMEEVAFGLELSGVNFIWVARFPKGEEQNLEDVLPKGFLERVGDRGRVLDHLVPQAHILNHKSTGGFISHCGWNSVMESIDFGVPIIAMPMQWDQPINARLLVELGVAVEIRRDEDGRIHRDEIAQVLKDVISGPTGEILRANVRDIALRLRARREEEMNAAAEELLQLCRNRRALK

SEQ ID NO: 3 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFEKLLKEYIEASESDEEVSCLITDALWYFAQSVADKLNLRRLVLQTGSLFNFHCLVSLPKFDELGYLDPDDKTNLEKPAEGFPMLKVKDIKRAYSHWQESKPILEKMVKQTKASSGVIWNSFKELEESELETVWREIPAPSFLIPLPKHYRASSTSLLDTDRSVFQWLDTQAPESVLYVGFGSQSEVDEADFLKIADGLVKSGISFLWLVRPGFVKGSTWIEPLPEGYLGDKGRIVKSAPQQEVLAHGAIGAFWTHGGWNGTMESVCEGVPMIFSDFGLDQPLNARYMSEVLKVGVYLENGWDPDEIAAAIRYVMRDEAGKPIRENAKVLKQKADRSIMKGGSSYESLESLVSYISSL